(19) United States
(12) Patent Application Publication
(10) Pub. No.: US 2009/0182296 A1
(43) Pub. Date: Jul. 16, 2009

(54) Body Adhering Article

(76) Inventors: Melissa Jean Dennis, Appleton, WI (US); Adrienne Rae Loyd, Neenah, WI (US); Thomas Harold Roessler, Appleton, WI (US); Thomas Andrew Lutzow, Providence, RI (US); Aidan J. Petrie, Jamestown, RI (US); Daniel Joseph Nelson, East Greenwich, RI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 12/364,486

(22) Filed: Feb. 2, 2009

Related U.S. Application Data
(63) Continuation-in-part of application No. 11/890,093, filed on Aug. 3, 2007.
Application No. 11/890,093 is a continuation-in-part of application No. 12/005,793, filed on Dec. 28, 2007.

(51) Int. Cl. A61F 13/82

(52) U.S. Cl. 604/385.05; 604/385.03

(57) ABSTRACT

A personal care article comprises an absorbent structure configured for disposition adjacent a wearer's vulva region to absorb bodily fluids discharged by the wearer, and a shell for supporting the absorbent structure at the vulva region. The shell has a first side and a second side, the first side having a first area and a second area. The first area surrounds and bounds at least a portion of the second area. In addition, at least a portion of the first area comprises a body adhesive. Furthermore, the absorbent structure is present in the second area of the shell. In addition, the first area of the shell contacts to the wearer's skin and/or hair surrounding the vulva region and the second area does not contact the vulva when in use.

### CROSS REFERENCE TO RELATED APPLICATIONS

This application is a continuation-in-part of U.S. patent application Ser. No. 11/890,093 filed Aug. 3, 2007, and U.S. patent application Ser. No. 12/005,793 filed Dec. 28, 2007. Each of these applications is hereby incorporated by reference in its entirety.

### BACKGROUND

Personal care articles are well known in the art. Such articles can desirably be disposable. Such articles can also be absorbent and can absorb discharged bodily fluids from a user. Such absorbent articles generally comprise a fibrous mass or other absorbent structure which can absorb and hold body fluids. Similarly, it is well known that feminine care articles have been employed to absorb and hold liquids, such as urine and/or menses. A typical structure of an absorbent article includes a fluid impermeable backsheet, a fluid permeable topsheet and an absorbent core positioned between the backsheet and the topsheet. Prior absorbent articles have also included various other features to improve fluid handling, such as intake layers, distribution layers, retention layers and the like. In these absorbent personal care articles, the topsheet is the body-facing side of the absorbent article and the backsheet is the garment-facing side of the absorbent article.

Generally, the absorbent articles are held in place during use by using the wearer's waist and elastic materials in the waist portion of the articles, in the case of pant-like garments, such as diapers and training pants, or by attaching the absorbent article to the underwear or undergarment of a wearer, in the case of pads or liners. Current methods of attaching the absorbent article to the underwear or undergarment of a wearer include placing an adhesive on the garment-facing side of the backsheet, having optional flaps (also called wings or side panels) that extend from the longitudinal sides of the absorbent article which wrap around the crotch portion of the underwear or undergarment of the wearer and a combination of the adhesive and the flaps.

It has also been suggested to use an adhesive to adhere the absorbent article to the skin of the wearer. However, the design of these absorbent articles was essentially the same as the absorbent articles which were attached to the underwear or undergarment of the wearer. That is, the adhesive is applied to the body-facing surface of the topsheet of the absorbent structure. Alternatively, in another design, a portion of the backsheet of the absorbent structure was wrapped around and over the topsheet. This portion of the backsheet which is wrapped around and over the topsheet of the absorbent structure then becomes a body-facing surface. An adhesive is applied to the portion of the backsheet which is wrapped over the topsheet. While these designs were effective for adhering the absorbent article to the skin of a wearer, these absorbent articles were not comfortable for wearers to wear, since the shape and size of the absorbent articles were the same as those absorbent articles which were attached to the undergarment or underwear of the wearer.

Similarly, absorbent articles that are attached to the underwear or undergarment of a wearer can also be uncomfortable for the wearer. This is because during normal movement of the body, portions of the body place opposed forces on the undergarment, which may cause the undergarment to be bunched or twisted. When this occurs, any absorbent article attached to the underwear or undergarment may also become bunched or twisted, causing discomfort to the wearer of the absorbent article. For example, the presence and absence of pressure from the absorbent article on the inner thighs as the wearer moves, which is often described by wearers as feeling “like a diaper”, is one source which compromises comfort for wearers of conventional absorbent articles, including liners, ultra-thin absorbent pads and maxi pads. In addition, the movement of the wearer or deformation of the underwear while being worn may also cause the absorbent article to have a poor fit against the body of the wearer, which could result in leaks from the absorbent article.

Another disadvantage of conventional absorbent articles is that the silhouette or outline of the absorbent article may be visible to others through the clothing of the wearer. Even currently available ultra-thin absorbent articles may be visible through tight fitting outer clothing of a wearer. Therefore, conventional absorbent personal care articles do not always provide discretion for wearers.

There is a need in the art to provide wearers of absorbent articles with a discrete article which can function as an undergarment if desired, is as easy to use as a conventional pad and is comfortable to wear and will effectively prevent or reduce premature leakage during use.

### SUMMARY

In response to the needs discussed above, an absorbent composition of the present invention provides a body-adhering absorbent article which is capable of absorbing bodily fluids.

In one embodiment of the present invention, a personal care article comprises an absorbent structure configured for disposition adjacent a wearer's vulva region and a shell for supporting the absorbent structure at the vulva region. The shell has a first side and a second side, the first side having a first area and a second area. The second area of the shell has a second area circumference, and the first area of the shell surrounds and bounds at least 1% of the second area circumference of the shell. At least a portion of the first area comprises a body adhesive for adhering the shell directly to the wearer. In addition, the absorbent structure is present in the second area of the shell. Furthermore, the first area of the shell contacts to the wearer's skin and/or hair surrounding the vulva region.

In further aspects, the first area of the shell surrounds and bounds between 25%-100% of the second area circumference. In still other aspects, the shell comprises a woven web, nonwoven web, a gel, a film, a sheet of a polymeric material, a foam, or a laminate. In yet other aspects, the shell comprises a silicone material.

In other aspects, the first area of the shell comprises adhesive properties, wherein the adhesive properties provide a means to attach the personal care article to the wearer's body. In still other aspects, the shell has a three-dimensional concave shape such that the first side of the shell has an inward curving surface. In yet other aspects, the shell is a unitary structure.

In some aspects, the personal care article further comprises an anterior portion and a posterior portion, wherein the posterior portion is adapted to attach to the body of a wearer between the vulva region and the coccyx of the body of the wearer and the anterior portion is adapted to attach to the mons veneris region of a wearer. In further aspects, the posterior portion further comprises a protrusion extending upward from the first side of the shell. In other aspects, the shell is breathable. In still other aspects, the shell comprises material that is selected from extensible or elastically extensible materials. In yet other aspects, the absorbent structure is attached to the shell via an attachment means selected from at least one of a snap, a hook-and-loop material, a line of adhesive or a spot bond. In still other aspects, the absorbent structure is at least partially free-floating.

In other aspects, the absorbent structure is sized and shaped to cover the labia majora of a wearer. In still other aspects, the absorbent structure further comprises at least one of a body-side liner, a backsheet, a surge layer or a transfer layer.

In some aspects, the personal care article further comprises a release sheet. In other aspects, the shell includes at least one of a printed color, a print design, a texture, a micro-embossing, a printed message or a set of instructions. In still other aspects, the personal care article further comprises a positioning aid. In yet other aspects, the personal care article further comprises a removal aid.

In other aspects, the personal care article is selected from a pantiliner, sanitary napkin, maxi-pad or an incontinence article. In still other aspects, the absorbent structure is integrated into the shell. In yet other aspects, the first area of the shell is liquid permeable and the second area of the shell is liquid impermeable. In still other aspects, the shell comprises an absorbent material. In yet other aspects, the personal care article is part of a personal care system.

In another embodiment of the present invention, a personal care article comprises a shell for attachment to the vulva region of a female wearer. The shell has a first side and a second side, the first side having a first area and a second area. At least a portion of the first area comprises a body adhesive for adhesively attaching the shell directly to the wearer's skin and/or hair surrounding the vulva region and the second area of the shell is not directly attached to the wearer's skin. In addition, the second area provides a barrier between the vulva and the wearer's clothing when in use. In other aspects of this embodiment, the personal care article further comprises an attachment means for attaching an absorbent structure. In still other aspects, the personal care article further comprises an absorbent material. In yet other aspects, the shell is selected from a nonwoven web, a gel, a film, a sheet of a polymeric material or a foam. In still other aspects, the shell is breathable and liquid-impermeable.

Numerous other features and advantages of the present invention will appear from the following description. In the description, reference is made to exemplary aspects of the invention. Such aspects do not represent the full scope of the invention. Reference should therefore be made to the claims herein for interpreting the full scope of the invention. In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

### BRIEF DESCRIPTION OF THE DRAWINGS

The foregoing and other features, aspects and advantages of the present invention will become better understood with regard to the following descriptions, appended claims and accompanying drawings where:

FIGS. 1A and 1B each show a top view of an aspect of an article of the present invention.

FIG. 2 shows a side cross-sectional view of an aspect of an article of the present invention.

FIG. 2A shows a cross-sectional side view of an article of the present invention with a laminate shell.

FIG. 3 shows a side view of another aspect of an article of the present invention wherein the shell has a concave shape.

FIG. 4 shows a cross-sectional side view of an article of the present invention with the absorbent core integrated into the shell.

FIGS. 5, 5A and 5B each show a top view of an aspect of an article of the present invention having a different shell shape.

FIG. 6A shows a bottom view of an aspect of an article of the present invention where only a portion of the absorbent structure is positioned over the shell.

FIG. 6B shows a top view of an aspect of an article of the present invention where only a portion of the absorbent structure is positioned over the shell.

FIG. 6C shows a cross-sectional view taken along sectional line 6C-6C.

FIG. 7 shows a top view of an aspect of an article of the present invention wherein the body adhesive is applied in an open pattern.

FIG. 7A shows a cross-sectional view taken along sectional line A-A.

FIGS. 8A, 8B and 8C each show an article of the present invention having a release sheet applied thereto.

FIG. 9 shows a top view of another article of the present invention having a design for attachment to a specific area of the body.

FIG. 10 shows a cross-section view of FIG. 9 along lines 9-9.

FIG. 11A and FIG. 11B show aspects of the present invention with placement guides.

FIG. 12 shows a perspective view of another embodiment of an article of the present invention.

FIG. 13 shows an exploded perspective of the article.

FIG. 14 shows a top view of the article.

FIG. 15 shows a bottom view of the article.

FIG. 16 shows a side view of the article.

FIG. 17 shows a side cut-away view of the article taken along line 19-19 of FIG. 14.

FIG. 18 shows a top view of a shell of the article.

FIG. 19 shows a top view of an absorbent structure of the article.

FIG. 20A shows a perspective view of another aspect of an article having a shell and an absorbent structure.

FIG. 20B shows a perspective view of another aspect of an article having a shell and an absorbent structure.

FIG. 20C shows a perspective view of another aspect of an article having a shell and an absorbent structure.

FIG. 21 shows a perspective view of the article of the present invention having a 3-dimensional absorbent structure.

FIG. 21A shows a cross-section view taken along line E-E of FIG. 21.

Repeated use of reference characters in the present specification and drawings is intended to represent the same or analogous features or elements of the present invention. The drawings are representational and are not necessarily drawn to scale. Certain proportions thereof may be exaggerated, while others may be minimized.

### DEFINITIONS

It should be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

It should be understood that the term “absorbent product” or “absorbent article”, as used herein, refers to any article used to control bodily fluids that are configured to absorb and retain bodily exudates, including urine, blood, menses, and other bodily discharges, such as sweat and vaginal secretions resulting from sexual activity and the like. In addition, the term is intended to include odor absorbing articles.

As used herein, the term “absorbent structure” is intended to mean a configuration of an absorbent material which allows fluids to be absorbed by the absorbent material. As used herein, an absorbent structure of the present invention does not include the shell.

As used herein, the term “attach” and its derivatives refer to the joining, adhering, connecting, bonding, sewing together, or the like, of two elements. Two elements will be considered to be attached together when they are integral with one another or attached directly to one another or indirectly to one another, such as when each is directly attached to intermediate elements. “Attach” and its derivatives include permanent, releasable, or a refastenable attachment. In addition, the attachment can be completed either during the manufacturing process or by the end wearer.

As used herein, “body-facing” means that surface or side of the article which is intended to be disposed toward or placed adjacent to the body of the wearer during ordinary use. The term “garment-facing” means that surface or side that is on the opposite side of the article from the body-facing surface or side. The garment-facing surface is an outward surface of the article and is intended to be disposed to face away from the wearer's body during ordinary use. The garment-facing surface is generally arranged to face toward or placed adjacent to the wearer's undergarments or clothing when the article is worn.

As used herein, the term “connected” is intended to mean directly connected and indirectly connected. By directly connected, it is intended that the connected elements are in contact with one another or affixed to one another. By indirectly connected, it is intended that one or more intervening or intermediate elements are between the two elements which are secured or “connected” together. The intervening elements may be affixed.

As used herein, the term “disposable” is used herein to describe personal care articles that are not intended to be laundered or otherwise restored or reused as a personal care article after a single use.

As used herein, the term “elastically” means that property of a material or composite by virtue of which it tends to recover its original size and shape after removal of a force causing a deformation. Suitably, an elastically extensible material can be elongated by at least 50 percent of its relaxed length and will recover, upon release of the applied force, at least 40 percent of its elongation.

As used herein, the term “liquid impermeable,” when used in describing a layer or multi-layer laminate means that liquid, such as urine, menses or bowel movement, will not pass through the layer or laminate, under ordinary use conditions, in a direction generally perpendicular to the plane of the layer or laminate at the point of liquid contact.

As used herein, the term “liquid permeable” refers to any material that is not liquid impermeable.

As used herein, the terms “nonwoven” and “nonwoven web” refer to materials and webs of material having a structure of individual fibers or filaments which are interlaid, but not in an identifiable manner as in a knitted fabric. The terms “fiber” and “filament” are used herein interchangeably. Nonwoven fabrics or webs have been formed from many processes such as, for example, meltblowing processes, spunbonding processes, air laying processes, and bonded-carded-web processes. The basis weight of nonwoven fabrics is usually expressed in ounces of material per square yard (osy) or grams per square meter (gsm) and the fiber diameters are usually expressed in microns. (Note that to convert from osy to gsm, multiply osy by 33.91.)

As used herein, the term “personal care article” includes, but is not limited to, articles such as diapers, diaper pants, baby wipes, training pants, absorbent underpants, child care pants, swimwear, and other disposable garments; feminine care products including sanitary napkins, wipes, menstrual pads, menstrual pants, pantiliners, panty shields, interlabials, tampons, and tampon applicators; adult-care products including wipes, pads such as breast pads, containers, incontinence products, and urinary shields; clothing components; bibs; athletic and recreation products; and the like.

As used herein, the term “polymer” generally includes, but is not limited to, homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic and random symmetries.

As used herein, the term “superabsorbent material” refers to a water-swellable, generally water-insoluble, hydrogel-forming polymeric absorbent material, which is capable of absorbing at least about 10, suitably about 30, and possibly about 60 times or more its weight in physiological saline (e.g., saline with 0.9 wt % NaCl). In contrast, the term “absorbent material” refers to a material that is capable of absorbing at least about 5 times or more its weight in physiological saline (e.g., saline with 0.9 wt % NaCl).

### DETAILED DESCRIPTION

The product of the present invention provides a personal care article which is designed to adhere to the body of a wearer in the area of the body of the wearer which may need bodily fluids controlled. In one particular aspect, the article is an absorbent article that is attached to the body of a female wearer to or around the vulva region of the body. By “to or around the vulva region”, it is meant adjacent regions of the body of a female including the pubic region and the perinea region up to and including the coccyx.

When applied to or around the vulva region of the female body, the personal care article may be used as a pantiliner, sanitary napkin or incontinence article. In addition, the article may be worn as an underwear substitute since the article of the present invention does not need underwear to hold the absorbent article in place. As an underwear substitute, the article provides protection to the vulva area by creating a barrier between the outer clothing and the vulva region of a wearer. Such an underwear substitute may or may not include an absorbent structure. For example, in some aspects where the article is worn as an underwear substitute that includes permanently connected absorbent structure. In other aspects, the article is a disposable article that can be worn without an absorbent structure, and/or can provide a means for attaching or changing out an absorbent structure during use. The article of the present invention can serve to protect the outer clothing of the wearer from bodily discharges from the vulva region, and optionally other regions (e.g., the perinea region) of the wearer's body. In other aspects, the article can serve to protect the sensitive skin and body features of the vulva region from roughness of the outer or adjacent garments or clothing, thereby preventing or alleviating irritation to the sensitive skin and body features of at least the vulva region.

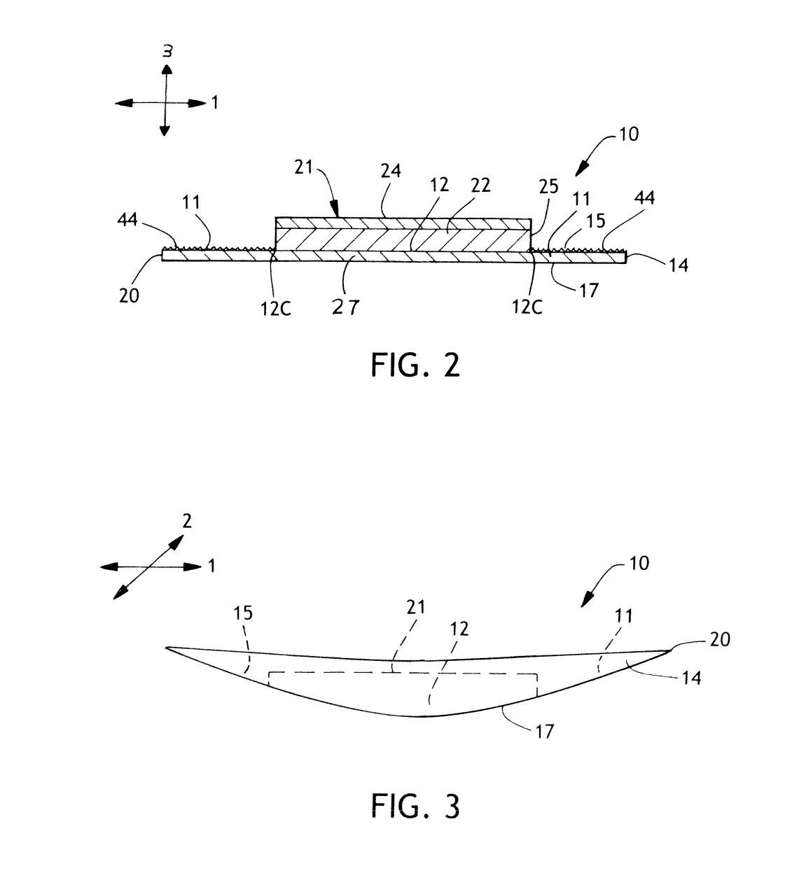

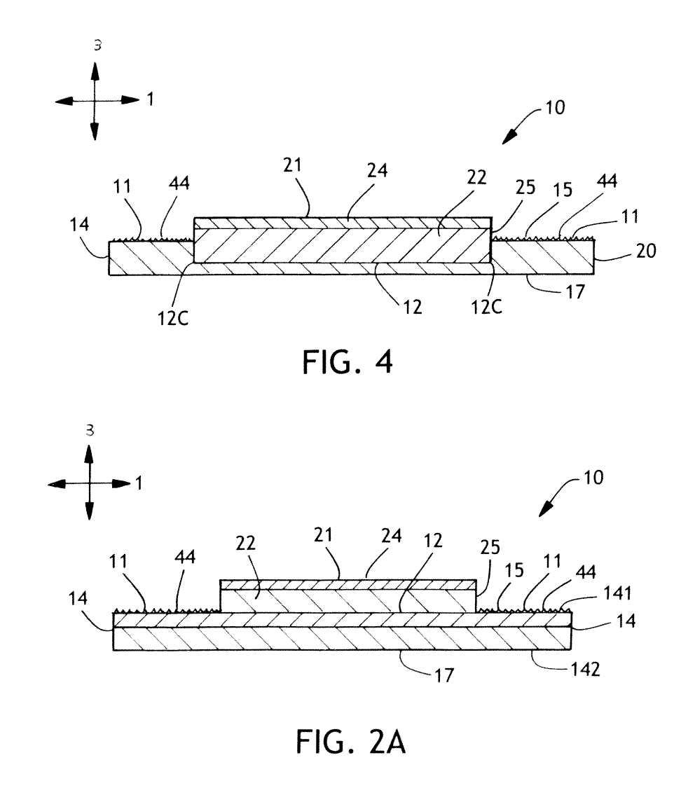

To gain a better understanding of the present invention, attention is directed to the Figures of the present specification. As is shown in the Figures, one aspect of the article 10 has a longitudinal direction 1 and a lateral direction 2. One component of the personal care article is a shell 14. This shell 14 has a first side 15 and a second side 17, as is shown in FIGS. 2 and 4. In some desirable aspects, the shell 14 is a single contiguous structure. However, it need not be. For example, in other aspects, the shell 14 can comprise two or more distinct structures that are directly or indirectly connected, as would be well understood by one skilled in the art. The shell 14 can help to provide the overall contour or silhouette of the article of the present invention. In addition, the shell 14 can also provide a surface for attachment or adhesion of the article 10 to the body of a wearer. In some desirable aspects, the shell 14 can also provide a surface for attachment or adhesion of an absorbent material or an absorbent structure, either prior to or during use.

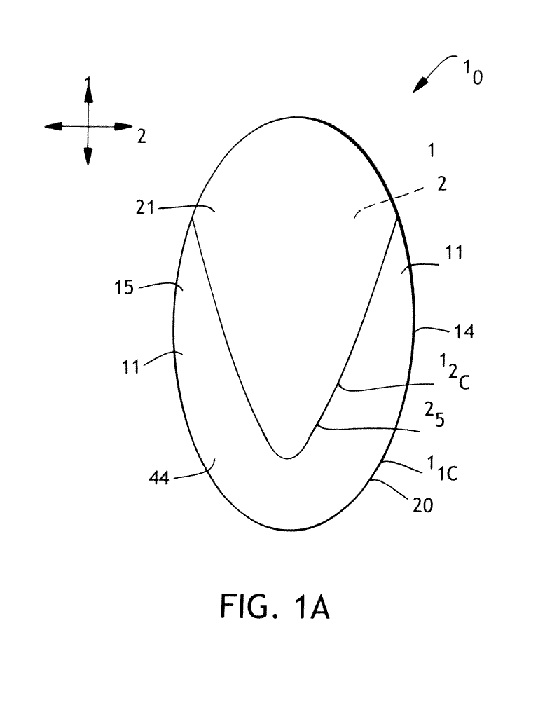

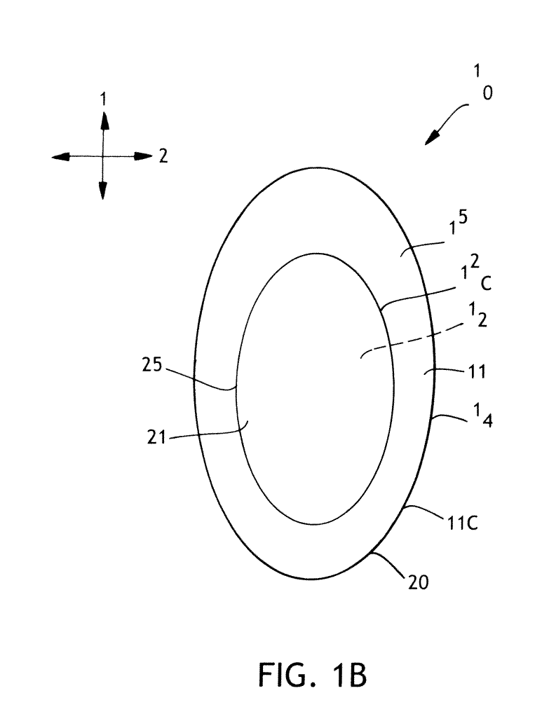

The first side 15 of the shell 14 is the body-facing side of the article 10 and the second side 17 of the shell 14 is the garment-facing side of the article. In some aspects, the first side 15 of the shell 14 has a first area 11 and a second area 12. The first area 11 surrounds or bounds the at least a portion of the second area 12, as is clearly shown in FIG. 1A. By “surrounds or bounds”, it is meant that at least about 1% of the circumference 12C of the second area 12 contacts the first area 11, such as at least about 25% of the circumference, or at least about 50% of the circumference, or at least about 75% of the circumference, or at least about 90% of the circumference 12C of the second area 12 is in contact with the first area 11. In a further aspect of the present invention, at least about 95% of the circumference 12C of the second area 12 is in contact with the first area 11. In still a further aspect of the present invention, the first area 11 completely surrounds the second area 12 of the shell 14 (i.e., 100% of the circumference 12C) as is shown in FIG. 1B.

In one aspect, the first area 11 of the first side 15 of the shell 14 is designed or adapted to contact, attach or adhere to the wearer's skin. In one particular aspect, the first area 11 of the shell 14 is designed or adapted to contact a wearer's skin surrounding the vulva region of the female torso when the article 10 is applied to the wearer. By “designed or adapted to contact a wearer's skin surrounding the vulva region of the female torso”, it is meant that the size and shape of the shell is such that the shell fits in the vulva region and possibly the surrounding pubic region and/or perinea regions and/or coccyx of the female torso. It is noted that the first area 11 may be a single contiguous area or may be two or more distinct areas.

As discussed above, the first area 11 is the portion of the first side 15 of the shell 14 which holds the article in place on the wearer. Generally, the shell 14 is desirably sized and shaped such that at least a portion of the first area of the shell only contacts and attaches or adheres to the skin and/or hair surrounding and proximate to the vulva area and/or the pubic and/or perinea and/or coccyx regions of the wearer. Accordingly, in addition to contacting the skin in the vulva, pubic perinea and/or coccyx regions of the wearer, the first area 11 of the first side 15 of the shell 14 may also, or alternatively, contact and attach or adhere to any hair in which may be present in those regions.

Generally, the second area 12 of the shell 14 is the portion of the shell 14 which provides absorbency to the article. That is, the second area 12 of the first side 15 of the shell 14 is any area of the first side 15 of the shell 14 to which an absorbent structure can be attached, or which has absorbent properties. In some aspects of the present invention, the second area 12 of the shell 14 has an absorbent structure 21 contained therein or attached to the shell 14 in the second area to form an absorbent article. In other aspects, the second area 12 of the shell 14 has an attachment means for connecting or changing out an absorbent structure during use. It is noted that the second area 12 may be a single contiguous area or may be two or more distinct areas. In some aspects, it may be desirable that the second area 12 is a single contiguous area from an ease of manufacturing standpoint.

In some aspects of the invention, the second area 12 of the shell 14 may include an absorbent material 27 applied to the surface of and/or integrated into the shell 14, such that the second area 12 of the shell is absorbent without the presence of an additional absorbent structure, such as a separate absorbent structure 21, to form an absorbent article. For example, the second area 12 of the shell 14 may have an absorbent material 27 coated or impregnated into the shell material. In further aspects, the second area 12 can include both a separate absorbent structure 21 and an absorbent material 27 coated onto and/or integrated into the shell 14. In still further aspects, the first area 11 of the shell 14 can at least partially include an absorbent material 27 coated onto and/or integrated into the shell. In other words, the absorbent material 27 can be present in at least a portion of the first area 11. Other such variations or combinations of absorbent structures and absorbent materials with respect to the first area 11 and the second area 12 will be apparent to those skilled in the art. In aspects which include the presence of an absorbent structure 21, such structure can be attached to the shell 14 using methods well known in the art, including adhesives, mechanically bonding the absorbent structure 21 to the shell 14 using bonding means such as ultrasonic bonding, heat and pressure bonding and the like, which are discussed in more detail below.

To gain a better understanding of the vulva region and surrounding regions of the female body, a general description of the anatomical structures can be found in The Illustrated Running Press Edition of the American Classic Gray's Anatomy (1974) by Henry Gray and Structure and Function in Man (1974) by Stanley W. Jacob, M.D., F.A.C.S. and relevant portions are included herein by reference. The general form can be found in Anatomy for an Artist: Elements of Form by Eliot Goldfinger and relevant portions are included herein by reference. The general description of the pubic hair covering these regions can be found in Woman's Body: A Manual for Life and relevant portions are included herein by reference.

The female anatomical structures to be described include the leg and the lower torso. The external anatomical structures of the lower torso include the gluteal region and the perineum region. The gluteal region includes the buttocks and the anus. The anatomical structure involved on the leg is the medial surface of the upper thigh.

The gluteal region includes generally the buttocks and anus and is typically bound in front by the line of the buttocks and the gluteal folds, in the back by the sacral triangle and the sides by lines extending through the greater trochanters. The shape of the gluteal region is roughly hemi-spherical and convex and is determined by a series of muscles, including the gluteus maximus and a series of fat pads including the posterior gluteal fat pad. The line of the buttocks separates the gluteal region and the perineum region.

The upper thigh region includes typically the right and left thigh and is typically bound on top by the thigh lines and the sides by the front and back of the leg. The thigh lines are two lines that are on either side of the labia and each of the lines runs along the line of the inguinal ligment to the gluteal folds and marks where the upper thigh meets the lower torso. The shape of the region is roughly a portion of a tapered cylinder and convex, and is shaped by a series of muscle groups including the gracilis, pectineus, adductor longus, adductor brevis, and adductor magnus and series of fat pads including the inner thigh fat pad.

The perineum region, which extends from the inferior outlet of the pelvis to the bony structure of the coccyx, is comprised of two divisions, the urogenital triangle and the anal division or obstetrical perineum. The region includes the external organs of reproduction; the mons pubis, labia majora and minora, clitoris, meatus urinarius and the opening to the vagina. The region is generally bound in front by the lower abdominal line, on the sides the thigh lines, and in the back the line of the buttocks. The abdominal line is a line that passes across the top of the pubis. The lines of the buttocks are lines that connect the thigh lines to the gluteal cleft. For convenience in describing the form and created spaces in the perineum region, this region will be subdivided into three regions including an anterior region including the mons pubis, a central region including the labia majora and minora, and posterior region. The anterior region is bound in front by the lower abdominal line, in back by anterior commissure, and on the sides by line of the labia. The central region is bound in front by the anterior commissure, in the back by the posterior commissure, and on the side by the line of the labia. The posterior region is bound in front by the line of the labia, in the back by the lines of the buttocks, and on the sides by the thigh line.

The vulva region includes the female external genitalia and generally includes the anterior and central regions of the perineum. The mons pubis [or veneris] is generally a rounded eminence in front of the symphysis pubis, formed by a collection of fatty tissue including the pubic fat pad beneath the integument and is generally covered with pubic hair. The labia majora are generally two prominent longitudinal cutaneous folds extending downward from the mons veneris to the anterior boundary of the perineum, and generally enclosing the common urinary-sexual opening. The space between the two folds is the labial cleft. Each labium has generally two surfaces, an outer, which is pigmented and covered generally with strong, crisp pubic hairs, and an inner within the labia cleft, which is smooth and is beset with large sebaceous follicles and is continuous with the genito-urinary mucous tract; between the two there is considerable quantity of areolar tissue, fat including the labia fat pad, and tissue besides vessels, meeting the anterior commissure. Posteriorly they are typically not joined, but generally appear to become lost in the neighboring integument, terminating close to, and nearly parallel with each other. Together with the connecting skin between them, they form the posterior commissure or posterior boundary of the vulval orifice. The interval between the posterior commissure and the anus constitutes the perineum region. The fourchette is the anterior edge of the perineum, and between it and the hymen is a depression, the fossa navicularis. The line of the labia separates the labia and the perineum region.

The labia minora are two small cutaneous folds, situated generally within the labia majora, and extending from the clitoris obliquely downward, outward, and backward on each side of the orifice of the vagina.

The form of the perineum, gluteal, and upper thigh regions combine to form a very intricate skin topography and spaces. The roughly two-hemispherical-like forms of the buttocks, the roughly tapered-cylinder-like form of the upper thigh, split-teardrop-like form of the vulvar region create intricate generally convex topography with intersections to form a series of recesses. The generally convex topography of the buttocks, the vulvar region, and upper thigh join to create spaces including two inner thigh grooves along two thigh lines, a depression in the posterior perineum region and a cleft extending through the labia and gluteal clefts. The grooves, depression, and cleft are like interconnected recesses in the topography. The central region general has lateral sides separated by a distal surface created by the labial cleft and includes the labial cleft.

Pubic hair generally cover some of these regions and fill in a portion of these recesses especially the labial cleft and the portion of the groove of the thigh parallel to the labial cleft to create a hair surface topography. The hair topography is the surface topography of an imaginary distal surface created by the hair. The depression of the perineum, thigh groove parallel to the gluteal cleft, and the gluteal cleft generally has little or no pubic hair. The skin topography combines with the hair topography to create an overall body topography.

This intricate space created by the intricate body form in this region of the body varies between women in both size and form, and varies with the position and movement of the women. Some of these variations are summarized in “Female genital appearance: ‘normality’ unfolds” by Jillian Lloyd et. al., BJOG: An International Journal of Obstetrics and Gynecology, May 2005, Vol. 112, pp. 643-646 and is included herein by reference.

The shell 14 of the article 10 of the present invention may be prepared from a variety of materials. The shell may include a layer constructed of any material which will provide the desired performance of the article. In some aspects, the shell material can be operatively liquid impermeable, while in other aspects, the shell material can be operatively liquid permeable. In still other aspects, the shell material can include combinations of liquid permeable and liquid impermeable materials, such as to form regions in the x-y and/or z-direction, for example. In one aspect, the shell 14 comprises liquid permeable material in the first area 11 of the shell 14, and liquid impermeable material (or materials that are treated to be liquid impermeable) in the second area 12 of the shell 14.

In general, the shell 14 can include a polymeric film, a woven fabric, a nonwoven fabric, a foam or the like, as well as combinations or composites thereof. In some aspects, the shell 14 may include a laminate structure, such as a polymer film laminated to a woven or nonwoven fabric, for example. In this particular feature, the polymer film can be composed of polyethylene, polypropylene, polyester, silicone or the like, as well as combinations thereof. A laminate shell 14 structure is shown in FIG. 2A, having an upper layer 141 and a lower layer 142, wherein the upper layer 141 is the body-facing side of the shell 14 and the lower layer 142 is the garment-facing side of the shell 14.

In some aspects, at least a portion of the first side 15 and/or the second side 17 of the shell 14 can be micro-embossed, textured, have a printed design, have a printed message to the consumer, and/or may be at least partially colored. In one particular aspect, the shell is colored and/or printed to resemble an undergarment.

In some aspects, the shell 14 can operatively permit a sufficient passage of air and moisture vapor out of the article 10, particularly out of the absorbent structure 21 while blocking the passage of bodily fluids and odors often associated with bodily fluids, particularly in the second area. Accordingly, other nonlimiting examples of a suitable shell material can include a breathable, microporous film, such as those described in, for example, U.S. Pat. No. 6,045,900 to Haffner et al., the entire disclosure of which is incorporated herein by reference in a manner that is consistent herewith. Still other shell materials include those that are extensible or elastically extensible. Suitable shell materials can also include various types of foams, including, but not limited to, thermoplastic foams, high internal phase emulsion (HIPE) foams and inverse high internal phase emulsion (I-HIPE) foams, and other suitable polymeric foams, including, but not limited to, those disclosed in U.S. Pat. No. 7,053,131 to Ko et al., U.S. Pat. No. 7,358,282 to Krueger et al. and U.S. Publication No. US2006/0148917 to Radwanski et al., which are incorporated herein by reference in a manner that is consistent herewith. One such example of a suitable foam is a polyurethane foam with a negative Poissons ratio invention. Materials used as backsheet materials are also suitable. Examples of extensible backsheet materials are described in U.S. Pat. No. 5,611,790, issued Mar. 18, 1997, to Osborn, III et al., herein incorporated by reference in a manner that is consistent herewith. Other materials that are inherently breathable, such as polyurethanes, may be used to form the shell 14.

In one particular aspect of the present invention, the shell 14 may be a laminate of a woven or nonwoven fabric with a silicone polymer, wherein the silicone polymer has adhesive properties. In this aspect, the second side 17 of the shell 14 can be the woven or nonwoven fabric and the first side 15 of the shell 14 can be the silicone polymer. One commercially available laminate is an OLEEVA FABRIC 1, available from Bio Med Sciences, Inc., having a place of business in Allentown, Pa., U.S.A. The OLEEVA fabric is a silicone sheeting having adhesive properties laminated to a fabric backing. In particular features of this aspect, the silicone sheeting can form the body-facing first side 15 of the shell material. Relating this particular structure to the Figures, in FIG. 2A, the silicone polymer is the upper layer 141 of the shell 14 and the nonwoven or woven layer is the lower layer 142 of the shell.

Bicomponent films or other multi-component films can also be used as the shell 14 material. In addition, woven and/or nonwoven fabrics which have been treated to render them operatively liquid-impermeable or liquid permeable can also be used as an effective shell 14 material. Another suitable shell material can include a closed-cell polyolefin foam, a polyurethane polymer material, a silicone polymer or other similar materials.

In some desirable aspects, silicone polymers having naturally occurring adhesive properties, or silicone polymers having a silicone adhesive layer applied thereto, can be used for the shell material. Such silicone polymers can allow the first area 11 of the shell 14 to adhere to the body of the wearer without the need of an additional adhesive. These materials may be laminated to another material, such that the other material is present on the second side 17 of the shell 14, which is the garment-facing side of the article 10, so that the adhesive nature of the silicone polymer does not adhere to the undergarments of the wearer. In another aspect of the present invention, the shell material may be prepared from an interpenetrating polymer network of two or more polymers. Generally, one of the polymers of the interpenetrating polymer network may be a silicone material. Examples of interpenetrating polymer networks are described in U.S. Pat. No. 5,759,560, issued to Dillion, which is hereby incorporated by reference in a manner that is consistent herewith.

The shell material should be selected such that the overall properties of the shell allow the shell material to move with the skin of the wearer during normal use and normal movements by the wearer during use. By “normal movement by the wearer” it is meant any movement that normally occurs during use of the article, including, walking, running, sitting, laying, standing, kneeling, riding a bicycle, exercising, lifting, playing sports, getting into and out of an automobile, and other similar movements made by wearers when wearing the article. The shell 14 should not be too rigid, such that the shell detaches from the skin of the wearer during use and the shell 14 should not be so flexible that the shell tends to twist and bunch during use. The shell 14 desirably has sufficient flexibility to conform to the skin of the wearer and become similar to an additional skin of the wearer. The shell also desirably has the ability to remain attached to the body of the wearer under dry, moist or wet conditions.

Generally, the shell material should have sufficient thickness to allow the shell 14 to mold to the body of the wearer, but not too thick that the shell 14 becomes uncomfortable for the wearer to wear. In addition, the shell 14 should not be so thin that it ineffectively forms a seal with the skin of the wearer when applied to the wearer, or becomes detached from the skin of the wearer during use and normal movement of the wearer during use, or that it does not adequately conform to the shape and skin of the wearer at the point of attachment to the wearer. Depending on the material used for the shell, the typical thickness of the shell is between about 0.03 mm and about 5.0 mm, such as between about 0.1 mm and about 3.0 mm, or between about 0.25 mm and about 3.0 mm to provide improved benefits. Again, the actual thickness used is dependent on several factors including rigidity of the material, the flexibility of the material and the ability of the material to assume the shape of the skin of the wearer at the location of use, which is typically the vulva region of a wearer, but can extend through the coccyx region in some aspects.

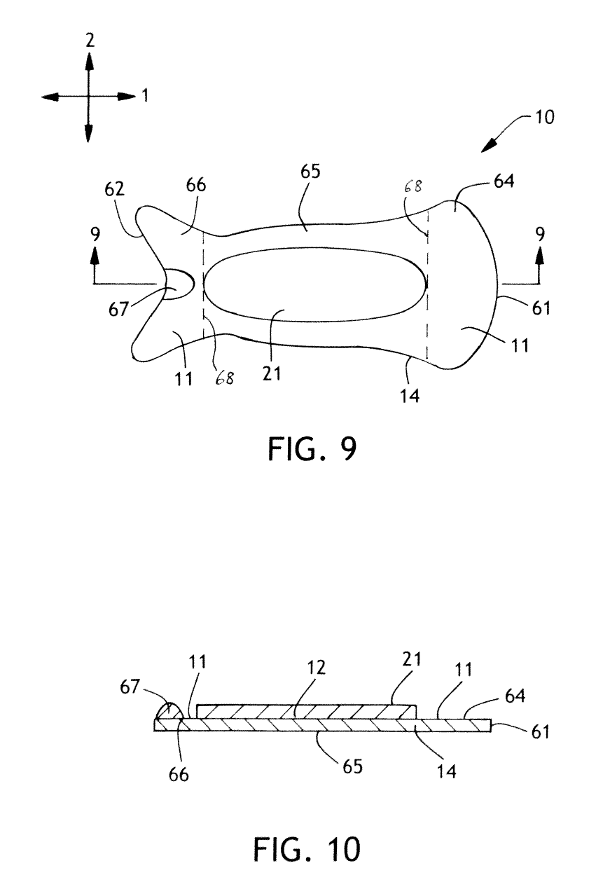

Generally, the second side 17 of the shell 14 forms at least a portion of the garment-facing side of the article 10 when worn by a wearer. In some aspects, the shell material can be selected such that the second side 17 of the shell 14 will freely move against the undergarment or clothing of a wearer. One way to achieve this result is to construct the second side 17 of the shell 14 to have a fairly low coefficient of friction, preferably the same or lower coefficient of friction as the adjacent surface of a garment with which the shell comes in contact with, such as an undergarment or other clothing. This will allow the second side 17 of the shell 14 to freely move against the undergarment or other clothing worn by the wearer. In this particular aspect, if the second side 17 of the shell 14 does not freely move against the undergarment or other clothing worn by the wearer, the article 10 may catch on the undergarment or clothing, which can result in the article being prematurely and undesirably removed from the wearer or may cause the article 10 to be shifted from its desired placement against the body of a wearer. However, in other aspects, it may be desirable that at least one or more portions of the second side 17 of the shell 14 have a coefficient of friction that is higher relative to the adjacent surface of a garment with which the shell comes in contact with. For example, as seen in FIG. 9, the shell 14 of the article of the present invention can be configured to have an anterior portion 64, a posterior portion 66 that is distal of the anterior portion, and a central portion 65 located between the anterior portion and the posterior portion. In this particular aspect, with respect to the adjacent surface of a garment with which the second side 17 of the shell 14 comes in contact with, at least a part of one of both distal end portions (i.e., anterior 64 and/or posterior 66 portions of the shell) can have a higher coefficient of friction, whereas, at least the central portion 65 of the second side 17 of the shell 14 can have a coefficient of friction that is the same or less than that of the adjacent surface of the garment.

In order to achieve the desired coefficient of friction on the second side 17 of the shell 14, the materials used to prepare the shell could be selected such that the second side 17 of the shell material will inherently have the desired coefficient of friction. Alternatively, the second side 17 of the shell 14 may be treated with a coating composition, such as polytetrafluoroethylene containing coating, a silicone containing coating or other similar coating having low coefficient of friction properties, for example.

In some aspects, the shell 14 can be made from a laminate of two or more materials such that the first side 15 of the shell 14 is prepared from a material which meets the needed properties of the first side 15, while the material selected for the second side 17 of the shell 14 meets the desired coefficient of friction such that the second side 17 will freely move against the undergarment or garment being worn by a wearer. In some aspects, the shell can be a unitary structure having desired surfaces, or regions on the same surface, of differing coefficients of friction. In other aspects, the shell may be assembled from multiple materials or sections (i.e., the shell is not unitary), each having a unique coefficient of friction.

The shell 14 of the article 10 may be flat or may have a three-dimensional shape. As is shown in FIG. 3, which is a side perspective view of the article 10, the shell 14 has a three-dimensional concave shape. Alternatively, as is shown in cross-sectional side views of FIGS. 2, 2A and 4, the shell 14 may have a generally flat shape. By providing the article 10 with a three-dimensional concave shape as is shown in FIG. 3, placement of the article may be easier for the wearer. Generally, the three-dimensional shape could be such that it closely matches the overall general curvature of at least a portion of the vulva region, such as when the article is used as a pantiliner, sanitary napkin, a feminine incontinence article or undergarment substitute. To form the shell 14 with a three-dimensional shape, the shell may be molded in any manner known to those skilled in the art, for example heat molding. The manner in which the three-dimensional shape is imparted to the shell 14 is not critical to the present invention.

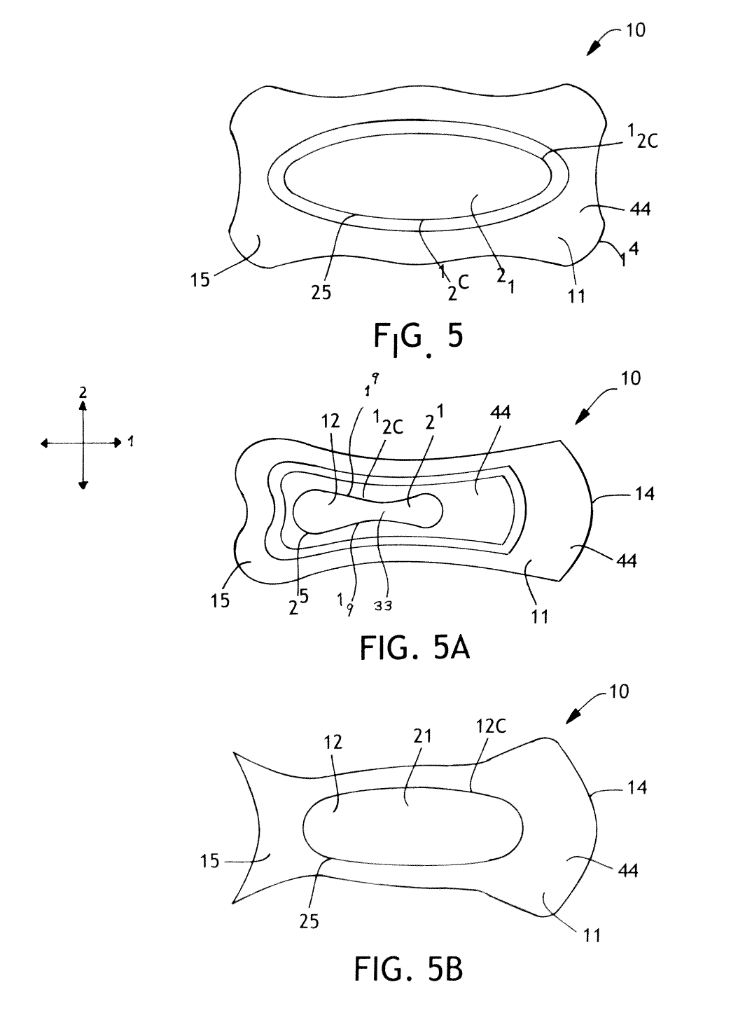

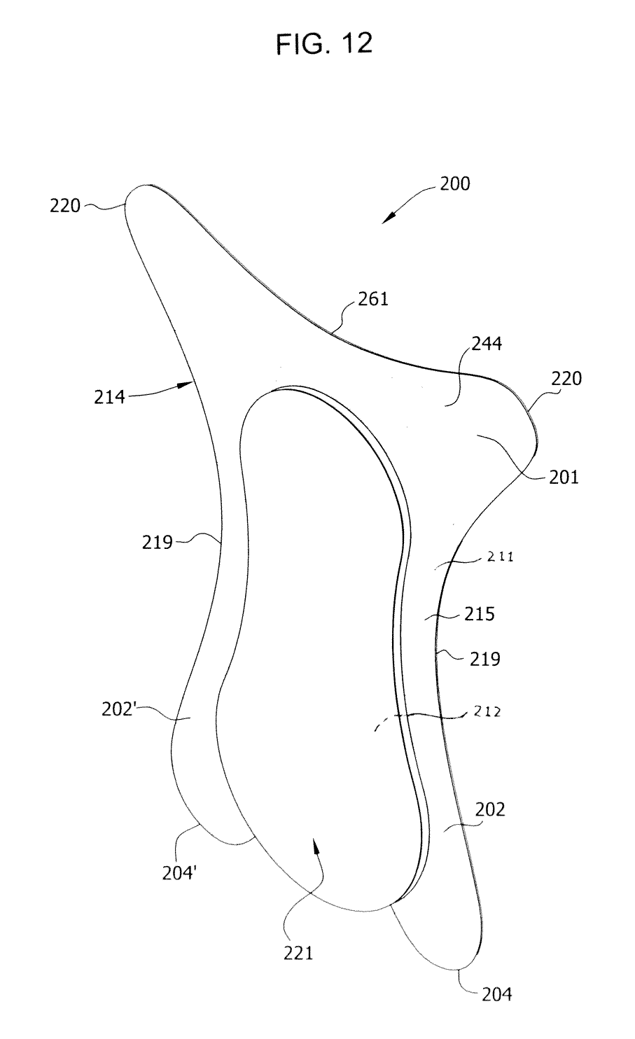

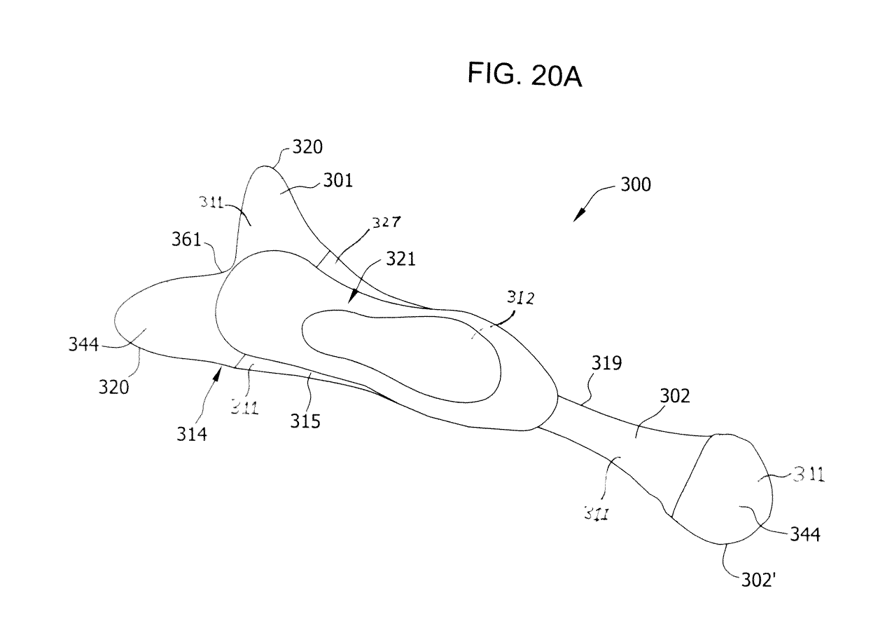

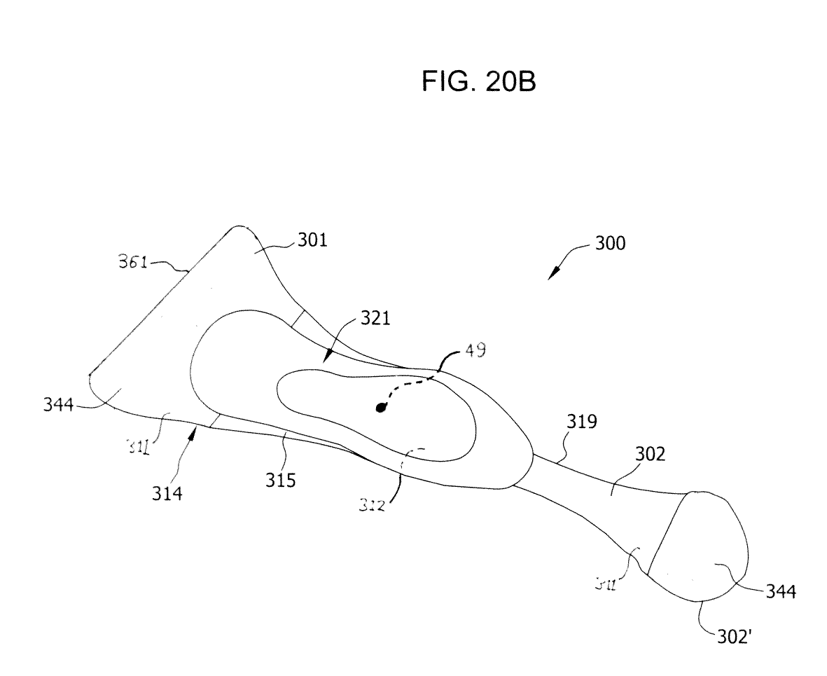

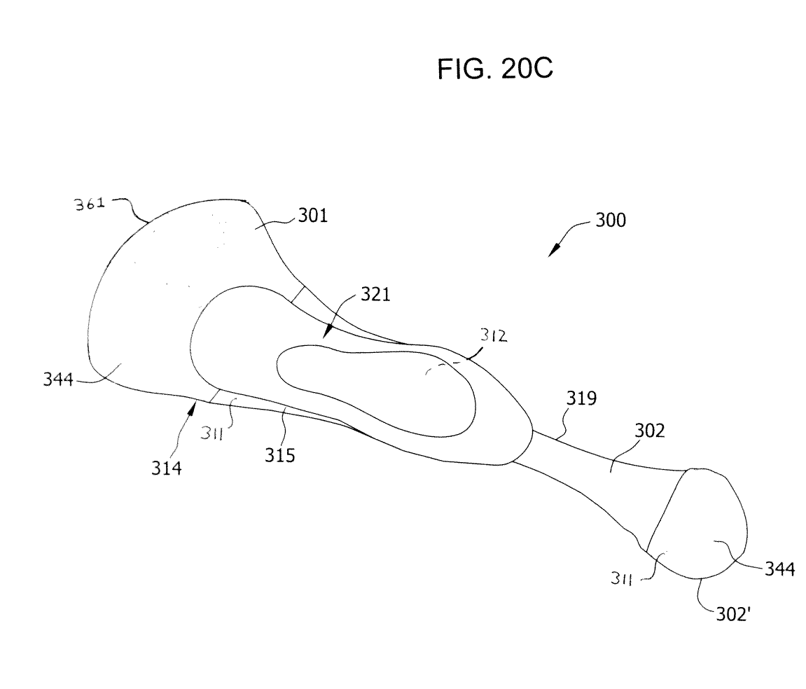

When the shell 14 is a generally flat shape, meaning that the shell does not have a third dimension other than thickness, the shell 14 is desirably constructed to be flexible enough such that the shell 14 can conform to the body of the wearer at the point of attachment. In addition to being flat, the overall shape of the shell 14 may be contoured, as is shown in FIGS. 5, 5A and 5B. In one aspect, the contour shape may be such that the narrowest point of the contour is in the crotch area of the shell 14 nearest the vulva region, as is shown in FIG. 5A. The contour shape shown in FIG. 5 is one of many possible shapes the shell 14 and article may be prepared. Other shapes may be used, such as those shown in FIGS. 12 and 20A-C. Still other shapes may be used without departing from the scope of the present invention. Generally, the shape selected should be such that the shell 14 and article 10 provide comfort and/or confidence to the wearer, while providing leakage protection to the wearer. It is noted that a contour shape may also be used in conjunction with a three-dimensional shell. Further discussion of the overall shape of the article may be found below.

In general, the shell may be any desired color and/or may be translucent. In addition, the shell may have a various finish, such as matte finish, satin finish or a smooth finish, or combinations thereof, for example. The particular finish color or translucency can be a matter of choice for the manufacturer of the article of the present invention. However, by providing a shell which is translucent may assist the wearer in placing the article 10 prior to use, since the wearer may be able to see where the article is placed compared to the genitalia of the wearer.

In some aspects, the article comprises an absorbent structure and/or an absorbent material to form an absorbent article. The absorbent structure 21 or absorbent material 27 are intended to absorb body exudates, including menstrual fluid, blood, urine, and/or other bodily fluids, such as sweat and vaginal discharges. In the case of an absorbent structure, the absorbent structure 21 has a longitudinal direction 1, a lateral direction 2, and a z-direction 3. This absorbent structure 21 may be a single layer or may be multiple layers. Typically, the absorbent structure 21 has an absorbent core 22, and can optionally comprise additional layers, such as a generally liquid-permeable topsheet 24 (i.e., topsheet) and/or a generally liquid-impermeable backsheet 23. This absorbent core 22 may contain one or more layers of absorbent materials, such as fibrous materials and/or superabsorbent materials for example. That is, the absorbent core 22 may be a single layer of absorbent materials or may be a multilayer structure. Each of the layers can contain similar materials or different materials. In some aspects, the article of the present invention is an absorbent article 10, where the materials that can be used to form the absorbent core 22 include those materials conventionally used in absorbent articles and includes materials, such as, for example, cellulose, wood pulp fluff, rayon, cotton, and meltblown polymers such as polyester, polypropylene or coform. Coform is a meltblown air-formed combination of meltblown polymers, such as polypropylene, and absorbent staple fibers, such as cellulose. A desired material is wood pulp fluff, for it is low in cost, relatively easy to form, and has good absorbency.

The absorbent core 22 can also be formed from a composite comprised of a hydrophilic material which may be formed from various natural or synthetic fibers, wood pulp fibers, regenerated cellulose or cotton fibers, or a blend of pulp and other fibers. One particular example of a material which may be used as the absorbent core is an airlaid material. The absorbent core 22 may have other properties including extensibility and elastic extensibility, which will allow the absorbent core to be extended or fit to a particular wearer. One example of extensible absorbent cores is described in U.S. Pat. No. 5,611,790 to Osborn, III et al., herein incorporated by reference in a manner that is consistent herewith. One example of elastically extensible absorbent cores is described in U.S. Pat. No. 6,362,389 to McDowall, herein incorporated by reference in a manner that is consistent herewith.

In some aspects, the absorbent core 22 also includes a superabsorbent material, in addition to or in place of the hydrophilic material, which increases the ability of the absorbent core to absorb a large amount of fluid in relation to its own weight. Generally stated, the superabsorbent material can be a water-swellable, generally water-insoluble, hydrogel-forming polymeric absorbent material, which is capable of absorbing at least about 10, suitably about 30, and possibly about 60 times or more its weight in physiological saline (e.g., saline with 0.9 wt % NaCl). The superabsorbent materials can be inserted as particles or in sheet form. In some aspects, the superabsorbent polymer may be applied as a liquid. The superabsorbent material may be biodegradable or bipolar. The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, and hydroxypropyl cellulose. The hydrogel-forming polymeric absorbent material may also be formed from synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof. The hydrogel-forming polymers may be lightly crosslinked to render the material substantially water insoluble. Crosslinking may, for example, be by irradiation or covalent, ionic, Van der Waals, or hydrogen bonding. Hydroxyfunctional polymers have been found to be good superabsorbents for sanitary napkins. Such superabsorbents are commercially available from BASF and Evonik Stockhausen, Inc., among others. Other types of superabsorbent materials known to those skilled in the art can also be used.

Generally, the absorbent core 22 will be positioned adjacent the shell 14, as is shown in FIGS. 2 and 2A for example. By “adjacent to the shell”, it is meant that the absorbent core 21 is directly in contact with the shell or may be separated by one or two additional layers or a construction or a bonding agent, such as pressure sensitive adhesive. In addition, the absorbent core 22 may be recessed into the shell 14 as is shown in FIG. 4.

In addition to the absorbent core 22, the absorbent structure 21 may have other additional layers which aid the absorbent core 22 in capturing and holding the bodily fluid into the absorbent core 22. These other optional layers, when present, in combination with the absorbent core 22 form an absorbent structure 21 of the article 10. There may be a single layer or multiple layers in addition to the absorbent core in the absorbent structure 21. Alternatively, the absorbent structure 21 may have a single layer, which is generally the absorbent core 22.

One particular example of an additional layer which may be used in addition to the absorbent core 22 in the absorbent structure 21 is a body-side liner or topsheet 24, which is generally a liquid permeable material, which allows bodily fluids to pass through the topsheet into the absorbent core. It is noted that the terms “body-side liner” and “topsheet” may be used interchangeable. The topsheet 24 also can provide a wearer with a dry feeling by separating the absorbent core 22 from the body of the wearer. That is, the topsheet 24 is placed between the absorbent core 22 and the body of the wearer such that the absorbent core 22 is between the topsheet 24 and the shell 14.

In the present invention, generally the topsheet 24 will only extend to the edge 25 of the absorbent core, as is shown in FIG. 2. However, the topsheet 24 may extend beyond the edge 25 of the absorbent core 22 and may be attached to the first side 15 of the shell 14. Generally, if the topsheet 24 extends beyond the absorbent core 22, the body-side liner will be attached to the first side 15 of the shell 14. Also, if the topsheet 24 extends beyond the absorbent core 22, the topsheet 24 will generally not cover the entire first area 11 of the first side 15 of the shell 14.

Optionally, the topsheet 24 may be formed from one or more materials. The body-side liner or topsheet 24 should be able to manage different body excretions depending on the type of product. In feminine care products, often the body-side liner or topsheet 24 must be able to handle menses and urine. In the present invention, the topsheet 24 may include a layer constructed of any operative material, and may be a composite material. For example, the body-side liner or body-contacting layer can include a woven fabric, a nonwoven fabric, a polymer film, a film-nonwoven fabric laminate or the like, as well as combinations thereof. Examples of a nonwoven fabric useable in the topsheet 24 include, for example, an airlaid nonwoven web, a spunbond nonwoven web, a meltblown nonwoven web, a bonded-carded web, a hydroentangled nonwoven web, a spunlace web or the like, as well as combinations thereof. Other examples of suitable materials for constructing the topsheet 24 can include rayon, bonded-carded webs of polyester, polypropylene, polyethylene, nylon, or other heat-bondable fibers, finely perforated film webs, net-like materials, and the like, as well as combinations thereof. These webs can be prepared from polymeric materials such as, for example, polyolefins, such as polypropylene and polyethylene and copolymers thereof, polyesters in general including aliphatic esters such as polylactic acid, nylon or any other heat-bondable materials. When the body-side liner is a film or a film laminate, the film should be apertured or otherwise be made to allow fluids to flow through the body-side liner to the absorbent core.

Other examples of suitable materials for the topsheet 24 are composite materials of a polymer and a nonwoven fabric material. The composite materials are typically in the form of integral sheets generally formed by the extrusion of a polymer onto a nonwoven web, such as a spunbond material. In some aspects, the body-side liner layer 24 can be configured to be operatively liquid-permeable with regard to the liquids that the article is intended to absorb or otherwise handle. The operative liquid-permeability may, for example, be provided by a plurality of pores, perforations, apertures or other openings, as well as combinations thereof, which are present or formed in the liner or body contacting layer. The apertures or other openings can help increase the rate at which bodily liquids can move through the thickness of the liner or body contacting layer and penetrate into the other components of the article (e.g. into the absorbent core 22). The selected arrangement of liquid permeability is desirably present at least on an operative portion of the topsheet 24 that is appointed for placement on the body-side of the article. The topsheet 24 can provide comfort and conformability, and can function to direct bodily exudates away from the body and toward the absorbent core 22. The topsheet 24 can be configured to retain little or no liquid in its structure, and can be configured to provide a relatively comfortable and non-irritating surface next to the body tissues of a wearer. In the present invention, the topsheet or body-facing surface of each article may be embossed, printed or otherwise imparted with a pattern.

In some aspects, additional layers or substrates, including for example, the liquid acquisition and distribution layer, also referred to as a surge or transfer layer, and an optional tissue layer are also incorporated into the absorbent structure 21 of the absorbent product 10, for example, between the topsheet 24 and the absorbent core 22. The distribution layer may be shorter than the absorbent core or have the same length as the absorbent core 22. The distribution layer serves to temporarily hold an insulting fluid to allow the absorbent core sufficient time to absorb the fluid, especially when a superabsorbent material is present.

In other aspects, the absorbent core, optional transfer layer and other optional components, such as tissue layers, may be free floating (unattached) between the shell 14 and the topsheet 24, and only are secured along only the peripheral edges thereof. Alternatively, the absorbent core 22, transfer layer, if present, and any other layer or component, if present, may be attached to one or both of the shell 14 and topsheet 24 and/or to each other.

In aspects wherein a liquid-impermeable backsheet is present, the backsheet 23 will typically be located on the garment-facing side of the absorbent core 22, such that the absorbent core is located between the backsheet 23 and the body of the wearer. The backsheet 23 may be prepared from a variety of materials which will function to provide desired properties. For example, the backsheet 23 may include a layer constructed of any operative material, and may or may not have a selected level of liquid-permeability or liquid-impermeability, as desired. In a particular configuration, the backsheet 23 may be configured to provide an operatively liquid-impermeable backsheet structure. The backsheet may, for example, include a polymeric film, a woven fabric, a nonwoven fabric or the like, as well as combinations or composites thereof. For example, the backsheet may include a polymer film laminated to a woven or nonwoven fabric. In a particular feature, the polymer film can be composed of polyethylene, polypropylene, polyester or the like, as well as combinations thereof. Additionally, the polymer film may be micro-embossed. Desirably, the backsheet 23 can operatively permit a sufficient passage of air and moisture vapor out of the article, particularly out of the absorbent core 22 while blocking the passage of bodily liquids. An example of a suitable backsheet material can include a breathable, microporous film, such as a HANJIN Breathable Backsheet available from Hanjin Printing, Hanjin P&C Company Limited, a business having offices located in Sahvon-li. Jungan-mvu.Kongiu-City, Chung cheong nam-do, Republic of South Korea. This backsheet material is a breathable film, which is dimple embossed and contains: 47.78% calcium carbonate, 2.22% TiO2, and 50% polyethylene.

In a particular feature, the polymer film can have a minimum thickness of no less than about 0.025 mm, and in another feature, the polymer film can have a maximum thickness of no greater than about 0.13 mm. Bicomponent films or other multi-component films can also be used, as well as woven and/or nonwoven fabrics which have been treated to render them operatively liquid-impermeable. Another suitable backsheet material can include a closed-cell polyolefin foam. For example, closed-cell polyethylene foam may be employed. Still another example of a backsheet material would be a material that is similar to a polyethylene film which is used on commercially sold KOTEX brand pantiliners, and is obtainable from Pliant Corporation, a business having offices located in Schaumburg, Ill., USA.

The absorbent structure 21 is generally attached to the first side 15 of the shell 14 in the second area 12 of the shell. The attachment may be in a permanent manner, meaning that the absorbent structure is generally intended not to be removable by the wearer of the article 10. Alternatively, the absorbent structure 21 may be constructed to be removable by the wearer, meaning that the absorbent structure 21 may be removed and replaced with another absorbent structure 21 by the wearer of the article 10, or be replaced with nothing at all. In some aspects, when the absorbent structure 21 is attached to the shell 14 in a permanent manner, meaning that the absorbent structure is not intended to be removed by the wearer, various bonding means can be used, such as a construction adhesive for example. Examples of useable construction adhesives include any adhesive which will effectively hold the absorbent structure 21 in place, so as not to be separated from the shell 14. Commercially available construction adhesives usable in the present invention include, for example, Rextac adhesives available from Huntsman Polymers, having a place of business in Houston, Tex., U.S.A., as well as adhesives available from Bostik Findley, Inc, having a place of business in Wauwatosa, Wis., U.S.A. Other means may be used to hold the absorbent structure 21 to the shell including bonding techniques known in the art, including, but not limited to, adhesive bonds, cohesive bonds, thermal bonds, ultrasonic bonds, embossing, crimping, entangling, fusing, hook and loop, or the like, and combinations thereof. In some aspects, the absorbent structure is connected to the shell via an adhesive that at least partially covers the area between the second area 12 of the shell and the absorbent structure 21.

When the absorbent structure 21 is removably attached, the absorbent structure 21 is held in place on the shell 14 by a means which will allow the wearer to remove the absorbent structure. One such means of holding the absorbent structure 21 is by using a pressure sensitive adhesive. Suitable pressure sensitive adhesives include, but are not limited to, any commercially available pressure sensitive adhesive. Examples of suitable pressure sensitive adhesives usable to removably hold the absorbent structure 21 in place on the shell 14 include pressure sensitive adhesives available from National Starch, having a place of business in Bridgewater, N.J., U.S.A. By providing an absorbent structure 21 which is removable, the shell 14 may be reused several times without the need to again replace the shell when the absorbent needs to be replaced. Also, by having a removable absorbent structure 21, the absorbent structure 21 can be selected by the wearer prior to use. This would allow the wearer to select an appropriate level of protection for a given day or allow the wearer to select a size or shape of the absorbent structure 21 which the wearer finds to be more comfortable or desirable.

Another advantage of having an absorbent structure 21 which is removable is that the wearer may be able to perform normal bodily functions, such as urination, without replacement of the entire article 10. By having the absorbent structure 21 which is removable, a wearer could remove the absorbent structure 21, urinate and return or replace the absorbent structure 21 only. This would alleviate the need of a wearer to have to replace the entire article 10 in order to form bodily functions. As another alternative, the absorbent structure 21 could be attached to the shell 14 in such a manner that the absorbent structure 21 is hinged with a hinging means.

To aid a wearer in replacing the absorbent structure 21, a placement aid may be present on the shell 14 and/or the absorbent structure 21. Suitable placement aids include the use of color, tactile indicators or any other means that would assist the wearer in replacing a removed absorbent structure.

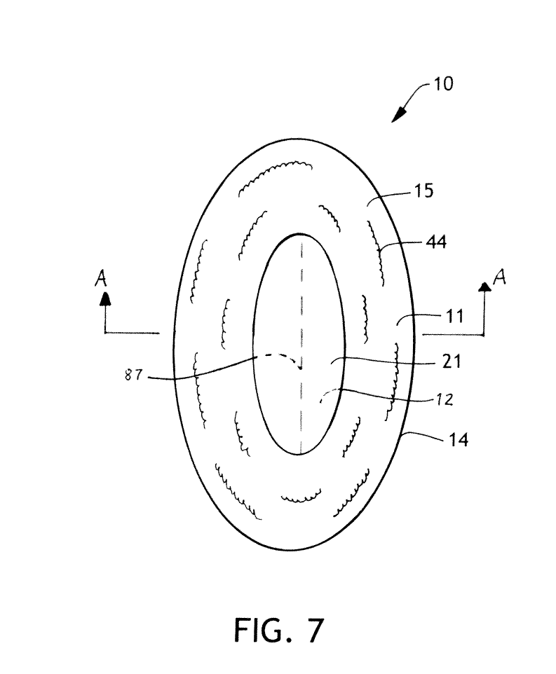

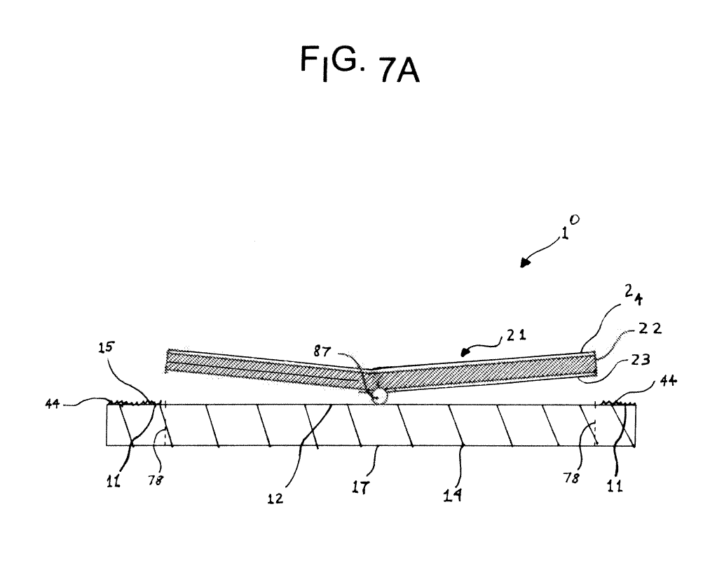

In one particular aspect, the absorbent structure 21 is connected to the second area 12 of the shell 14 via at least one line of adhesive 87, such as seen in FIG. 7 and FIG. 7A. (In the illustrated embodiment of FIG. 7A, the first area and second area are discerned by dashed line 78). Such a line of adhesive 87 can allow the absorbent structure 21 to be partially free-floating with respect to the shell 14, allowing the article to better conform to the wearer during movement and various changes in position of the wearer. In another particular aspect, the absorbent structure is connected to the shell 14 via at least one spot bond 49, such as seen in FIG. 20B. The spot bond 49 can provide additional ability for the absorbent structure 21 to be at least partially free-floating in both the x- and/or y-position to better conform to the wearer during movement and various changes in position of the wearer. In some aspects, the spot bond can be ideal when the absorbent structure is removable, such as when the spot bond is in the form of a refastenable attachment means, including, but not limited to, a pressure sensitive adhesive, a hook-and-loop material, snaps, and the like, and combinations thereof. In some aspects, the article can include multiple lines of adhesive, spot bonds, and combinations thereof. In further aspects, the lines of adhesives and/or spot bonds can be placed in particular locations to provide improved benefits, such as improved fit and contour of the article during movement of the wearer.

As stated above, the absorbent structure 21, when present, is located in the second area 12 of the shell 14 and on the first side 15 of the shell 14. The size and shape of the absorbent structure 21 may be varied depending on the intended use of the article and will be discussed in more detail below.

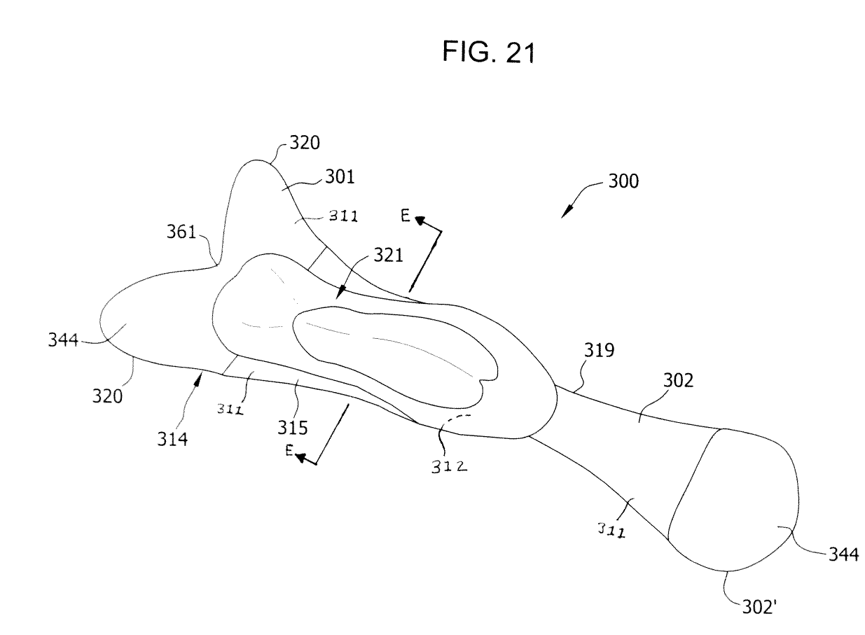

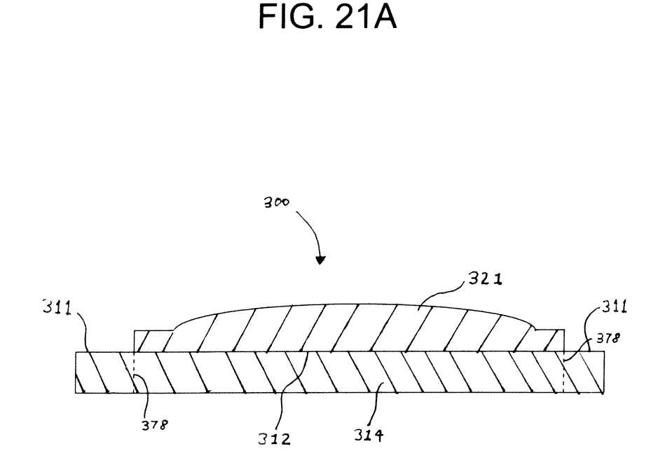

In some aspects, the absorbent structure 21 can be a relatively flat structure, as shown in FIGS. 2, 2A, and 4. Alternatively, the absorbent structure can have a three-dimensional shape other than a relatively flat shape, such as shown in FIGS. 21 and 21A (shown as element 321). The absorbent structure can have an anatomically correct shape such that the absorbent structure fits within the labia of the wearer. Anatomically correct shapes of absorbent are generally known to those skilled in the art and are generally found in the interlabial art field. The absorbent structure may be designed to be partially or fully interlabial. Alternatively, a three-dimensional shaped absorbent structure 21 may also be used in the article 10 which is designed not to fit within the labia majora of the wearer. That is, the absorbent structure 21 is positioned completely outside the labia during use.

The size, location and shape of the absorbent structure 21 may also be selected for an intended use. For example, in an overnight use, the absorbent structure 21 may be located further back on the wearer towards the perinea region of the wearer. In an overnight use, the absorbent structure may be larger than in a product intended for daytime use. In a daytime use, the absorbent structure will generally be centrally located in the vulva region.

In an alternative aspect of the present invention, absorbent material 27 is contained within the shell material. That is, the absorbent material 27 is an integral part of the shell 14 (i.e., integrated) and a separate absorbent structure 21 is not present. One way to achieve an integral absorbent structure is to have a shell 14 which is prepared from a material which is a laminate of two or more materials. The first side 15 of the shell 14 contains an absorbent material 27 within the body-facing side of the laminate. For example, superabsorbent particles or materials may be incorporated into the material making up the body-facing layer of the laminate. Another way is to place a very light coating onto the first side 12 of the shell material, wherein the coating contains superabsorbent particles or a liquid superabsorbent material. Of course other absorbent materials, other than superabsorbent materials, may be used in place of or in addition to the superabsorbent materials.

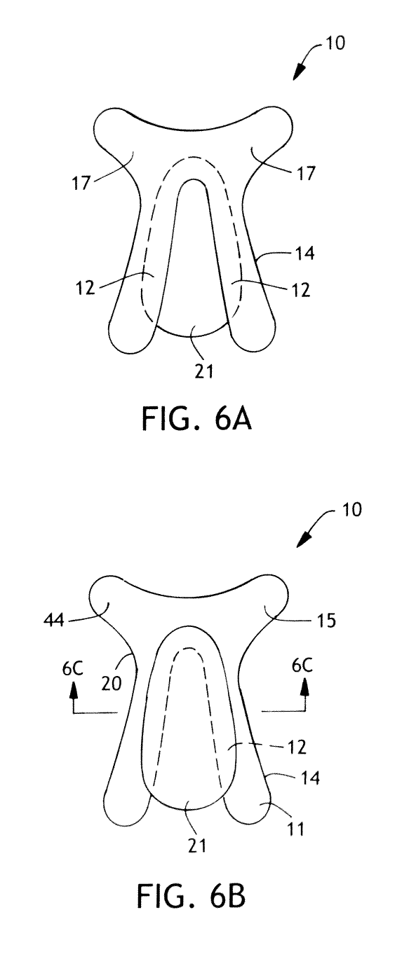

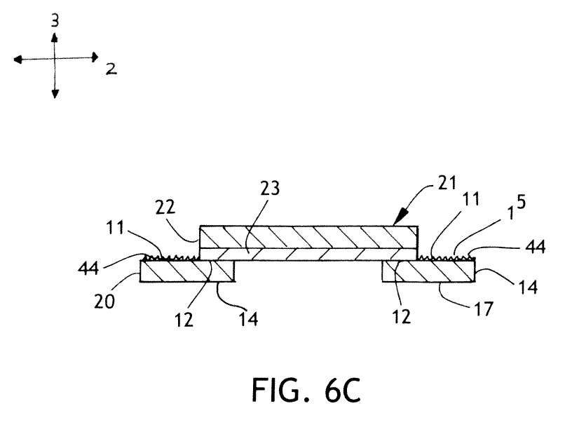

The absorbent structure 21 may be located entirely over the shell 14, as is shown in FIGS. 1, 2, 2A, 3, 4 and 5, meaning that the shell 14 material is located beneath the absorbent structure 14. Alternatively, the absorbent structure 21 may be positioned over the shell 14, such that only a portion of the absorbent structure 21 is over the shell 14. This configuration is shown in FIGS. 6A, 6B and 6C. FIG. 6A is a bottom view and FIG. 6B is a top view of an article 10 within the present invention. As can be seen only a portion of the absorbent structure 21 is positioned over the shell 14. FIG. 6C shows a cross-sectional view of the article 10 taken along line 6C-6-C in FIG. 6B. As with the other aspects of the present invention, the portion of the first side 15 of the shell 14 in which the absorbent structure is attached is the second area 12 of the shell 14. Surrounding the second area 12 is the first area 11 of the shell 14. The second side 17 of the shell 14 is the side of the article which faces the wearer during use. By having a personal care article with the structure shown in FIG. 6C, it is also beneficial for the absorbent structure to have a backsheet 23. The backsheet can serve to provide liquid impermeability to the absorbent structure 21, such that any fluids entering the absorbent core 22 will not flow through the core to the clothing of a wearer.

As is stated above, the first area 11 of the shell 14 serves to either directly or indirectly attach the article 10 to the body of a wearer. Stated another way, the shell 14 is the body attachment member and at least a portion of the first area 11 is the portion of the shell 14 which is attached to the body of the wearer. Depending on the material selected for the shell, the shell 14 may actively attach to the body of the wearer using electrostatic means; suction means, body adhesive means, frictional means and the like may be placed on the first area 11 of the shell 14 to attach the article to the body of a wearer. Electrostatic means can be used by selecting the shell material to be a material which has an affinity for the body of a wearer, such that the shell material “clings” to the body of the wearer. Examples of such materials include ethylene vinyl acetate, low density polyethylene and other similar materials known to those skilled in the art. Suction means may be achieved by shaping the shell to conform to the body of the wearer, much like a contact lens fits to the eye. Generally, suction means can be achieved by forming the shell 14 into a three-dimensional shape. Frictional means can be used by selecting a material or modifying a material to have a desired coefficient of friction to prevent slippage or detachment, such as for a shell member that provides for attachment in the gluteal groove.

A further desirable means to achieve body attachment is to place a body adhesive in the first area 11 of the shell 14. For example, a body adhesive 44 can be positioned on the first area 11 of the first side 15 of the shell 14. The body adhesive 44 contacts the skin and/or hair, if present, in the vulva region and possibly the pubic region and/or the perinea region and/or the gluteal region and/or the coccyx region of the wearer's body, thereby supporting and holding the article 10 against the body of the wearer during use. The body adhesive 44 can overlie a portion of the first area 11 or can overlie the entire first area 11 of the shell 14. Generally, the body adhesive 44 will be present on at least an outer portion or near the circumference 11C of the first area near the edge 20 of the article 10. As is shown in FIGS. 1A, 1B, 2, 2A, 4, 5B and 6B and 6C, the body adhesive 44 may cover the entire first area 11 of the article 10. Alternatively, the body adhesive 44 may be placed on a portion of the first area 11, as is shown in FIGS. 5 and 5A, and 20A-C (element 344). The body adhesive 44 may also be placed in a pattern of the first area 11. The body adhesive 44 can be applied to the first area 11 of the shell 14 using any known process including, but not limited to, inkjet printing, screen printing or extruding the body adhesive 44 from one or more nozzles, slot coating and the like.

Generally, any pressure sensitive adhesive known to those skilled in the art may be used, although preferably the pressure sensitive adhesive is not a known irritant to human skin and preferably the adhesive is not so aggressive that it causes pain to the wearer when the article 10 is removed from the skin. It is also desirable that the adhesive 44 is selected such that the adhesive does not leave a substantial amount of an adhesive residue on the surface of the skin of the wearer, when the article 10 is removed by the wearer after use. Particularly suitable pressure sensitive adhesive materials are disclosed in the commonly assigned U.S. Pat. No. 6,213,993 to Zacharias et al. and U.S. Pat. No. 6,620,143 to Zacharias et al., which are incorporated herein by reference in a manner that is consistent herewith. Other suitable adhesives are disclosed in U.S. Pat. No. 5,618,281 to Batrabet et al., which is incorporated herein by reference in a manner that is consistent herewith. Other known body adhesives, such as those described in U.S. Pat. No. 6,316,524 to Corzani et al., which is incorporated by reference in a manner that is consistent herewith, may also be used. Additional examples of suitable pressure sensitive adhesives include, hydrogels, hydrocolloids, acrylic based adhesives, and rubber based adhesives, such as KRATON based adhesives.

The body adhesive 44 may be positioned on the first area 11 of the shell 14 in an open pattern or a closed pattern. By “open pattern” it is meant that the adhesive can have an intermittent or discontinuous pattern which does not substantially encircle the entire first area 11. An example of an “open” pattern of the adhesive would be to have individual beads of adhesive applied in a discontinuous fashion. An open pattern of adhesive is shown in FIG. 5. “Closed pattern” means the adhesive 44 would encircle the entire second area 12 of the shell. In some aspects, the pattern of the body adhesive 44 will preferably substantially surround the absorbent structure 21 located in or on the second area 12 of the shell 14. As shown in FIGS. 1A, 2, 2A, 4, 5B, 6B and 6C, the body adhesive 44 is applied in a closed pattern, since the entire body adhesive is applied in a continuous fashion around the first area. An “open” pattern of the adhesive is shown in FIG. 7, which shows the body adhesive 44 applied in a discontinuous fashion. Additionally, the adhesive may be applied in particular portions of the first area 11, as is shown in FIGS. 5 and 5A. In some aspects, a closed pattern can be advantageous since the body adhesive 44 can form a seal with the body of the wearer which will assist in preventing leaks from the article 10. The body adhesive can form a dam, which may prevent leaks from the entire perimeter of the article.

In some aspects of the present invention, as is shown in FIGS. 1A, 2, 2A, 4, 5B and 6B and 6C, the body adhesive 44 may be placed on the entire first area 11, just outside of the absorbent structure 21. In other aspects of the present invention, as is shown in FIGS. 5 and 20A-C, the body adhesive 44, 344 may be placed along the outer portions of the first area 11 near the periphery of the shell 14. In some aspects, the body adhesive 44 may also be placed on the absorbent structure 21. Generally, however, the body adhesive 44 is confined to being placed on the first area 11 of the shell 14, since placing the body adhesive on an area of the article 10 which contacts the female genitalia such as the labia majora may cause discomfort to the wearer of the article.

In some aspects, the adhesive may be applied in a pattern of small discrete dots so as to leave numerous areas free from adhesive. Alternatively, the adhesive may be applied as a continuous bead, or may be applied as a series of semi-continuous beads. Other suitable adhesive patterns may be selected for applying the body adhesive 44 to the body-contacting first area 11 of the article 10. For example, adhesive patterns can be oval, swirls, various linear or non-linear arrays of adhesive longitudinally, and/or transversely oriented and reticulated webs having unobstructed interstices between the adhesive fibers or combinations thereof. As stated above, the adhesive patterns may be open or closed. The weights of adhesives are limited to less than about 800 g/m², and generally less than about 400 g/m². Generally, the weight of the adhesive is at least 20 g/m². Typically, the adhesive is applied in an amount of about 100 to about 400 g/m². The limitations on the basis weight of the adhesive are important to provide the correct adhesive characteristics for applying directly to the wearer's vulva region and optionally the pubic, perinea, gluteal and/or coccyx regions of the wearer's body. If the basis weight is too high, the article will have a sticky feeling or otherwise uncomfortable feeling. If the basis weight of the adhesive is too low, there may be insufficient adhesion to the body of the wearer.

Generally, the body adhesive 44 is applied in a manner which is symmetrical about the longitudinal axis 1 which bisects the article 10 and divides the article 10 into substantially equal portions. This symmetrical pattern provides the wearer a balanced feel when wearing the article 10. The symmetrical pattern also reduces the perception of any associated discomfort when the article 10 is removed from the body.

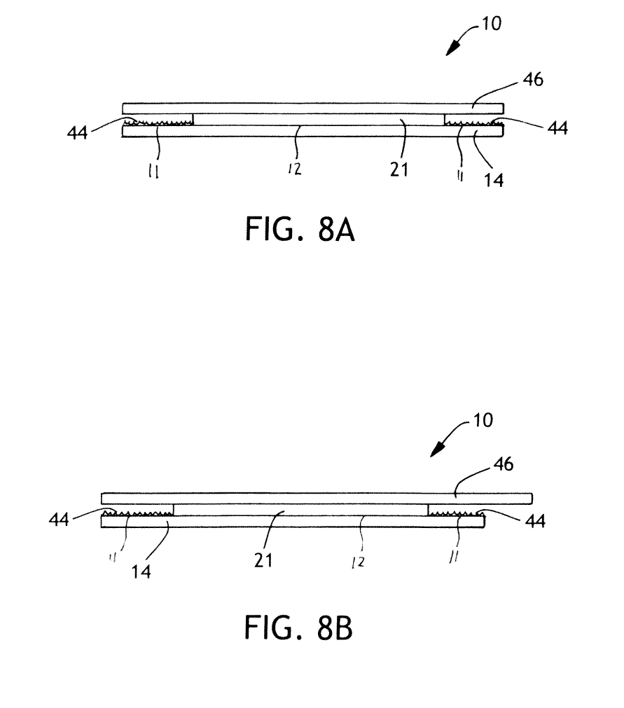

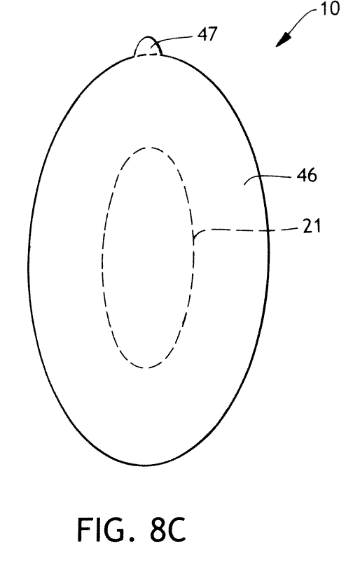

As is shown in FIGS. 8A and 8B, to protect the body adhesive 44, a peel sheet or release sheet 46 may be used to prevent the body adhesive 44 from becoming contaminated, thus losing its ability to stick to the body of a wearer and/or prematurely adhering to an unintended surface. Suitable materials for use as a peel strip 46 are well known in the art and are commercially available. Examples of suitable peel sheets or release sheets include, but are not limited to, a silicone coated Kraft paper, a silicone coated film or the like. Other release coatings include coatings containing polytetrafluoroethylene. The peel sheet or release sheet 46 may extend beyond one or both of the ends and/or sides of the shell, as shown in FIG. 8B. Alternatively, the release sheet 46 may be sized to only cover the body adhesive on the first area 11 of the shell 14, as is shown in FIG. 8A. In yet another aspect of the present invention, release sheet 46 may extend beyond the adhesive 44 at one or more locations, such as one of the ends or one of the sides of the shell 14 as is shown in FIG. 8C by providing the release sheet 46 with a tab 47 for the wearer to grasp to remove the release sheet 46 from the article 10 and the body adhesive 44 on the article. When the release sheet 46 extends beyond the adhesive 44, it is generally easier for the wearer to remove the release sheet 46 to place the article 10 for use.

Alternatively, the release sheet 46 may be provided with a pressure sensitive adhesive to hold the release sheet 46 in place when the article is devoid of an adhesive for body attachment. In this configuration, the release sheet serves to protect the absorbent structure 21 and first side 15 of the shell 14 from dirt and damage prior to use.

In another aspect, a release sheet may not be necessary. For example, the article may be rolled, folded onto itself or stacked upon each other. In these configurations, a release sheet is not needed. If rolled, the body adhesive 44 will generally contact the second side 17 of the shell 14. The body adhesive 44 should releasably stick to one second side of the shell by readily releasing when unrolled by the wearer. In addition, the body adhesive 44 should not leave a residue on the second side 17 of the shell. This should similarly occur when the articles 10 are stacked upon each other such that the body adhesive 44 of one article will attach the second side of the shell of a second article. In another possible configuration, the article 10 may be folded along the longitudinal axis 1 of the lateral axis such that the body adhesive 44 in one area comes into contact with body adhesive in another area. In the folded configuration, the body adhesive should be selected such that the body adhesive will release from itself when manipulated by a wearer.

The dimensions and shape of the shell 14 should be such that it is appropriately sized for its intended use. The same is true for the size and shape of the absorbent structure. Generally, the size and shape of the absorbent structure 21, when present, will dictate the size of the shell 14. The shape of the shell 14 is selected so that the article 10 will have a comfortable feeling for the wearer, thereby providing protection against leaks and preventing the article from becoming dislodged from the body of the wearer during wearing. In some aspects, the shell will be curved to fit the body of a wearer. In some aspects, the shell 14 also generally gives the article 10 its overall size and shape in the longitudinal 1 and lateral 2 directions. That is, the shell 14 can be generally longer and/or wider than the absorbent structure 21, as can be seen in the Figures. In other words, the shell 14 can be wider in the lateral direction 2 than the absorbent structure 21, and/or the shell can be longer in the longitudinal direction 1 than the absorbent structure 21. However, in some aspects, it is also possible for the absorbent structure 21 to be the same or longer and/or the same or wider than the shell 14.

When the article is intended for use as a pantiliner, a sanitary napkin or a feminine incontinence article, the shell 14 can be wider and longer than the absorbent structure 21 attached to the second area 12 of the shell 14. The absorbent structure can be at least as wide and as long as the labia majora of the wearer. As a result, to fit most women, the absorbent structure is longer in the longitudinal direction than it is wide in the lateral direction of the absorbent structure. Generally, for most women, the labia majora are generally between about 40 mm and about 70 mm in width and between about 80 mm and 150 mm in length. Ideally, the absorbent structure should be wider than the labia majora and slightly longer that the labia minora and slightly longer than or equal to the labia majora. Generally, the absorbent structure 21 should be between about 40 mm and 90 mm in width in the lateral direction and between about 95 mm and about 150 mm in length in the longitudinal direction. The shape of the absorbent structure 21 will generally tend to be oblong and may be an oval, a rectangle, teardrop shaped, hourglass shaped or racetrack shaped, for example. As can be seen in FIGS. 1A, 5, 5B, 6B,7 and 9, the absorbent structure 21 has a generally elliptical or oval shape to match the size and shape of the vaginal area of most women. An example of a teardrop shaped absorbent is shown in FIG. 1A.

Generally, the shape of the shell 14 may vary from a generally oval shape, as shown in FIGS. 1A and 1B to a shape which is a generally hourglass-like shape, shown in FIG. 5A. By generally hour glass shape, it is meant a shape in which the sides 19 of the shell 14 converge towards one another at a point along the longitudinal axis 1 of the shell 14 to form a narrowest portion 33 of the article. Generally, the hourglass-like shape provides a cut-out for the wearer's legs. By having an hourglass-like shape, the shell 14 will preferably not be attached to the legs of a wearer during use. This can provide additional comfort to the wearer of the article 10. The shape of the shell 14 should be selected such that the article 10 will be comfortable to wear, while providing very effective leakage protection to the wearer. The shell 14 and the absorbent structure 21 should preferably be capable of adapting to the curvature of a wearer's body during use. Other possible shapes for the shell 14 are also shown in FIGS. 5, 5A, 5B, and 6A, and 20A-C (shown as element 314). Other shapes not specifically shown may also be suitable.

In some aspects when the article is used as a sanitary napkin or an incontinence article, to obtain an effective attachment of the article to the wearer, generally the width of the shell 14 should be at least 10 mm on either side of the labia majora. Generally, the shell 14 of the article 10 will have a width, in the lateral direction 2, between about 50 mm up to 200 mm or more. Typically, the shell will be between about 60 mm and 120 mm at its narrowest point. This will allow the shell 14 to have a first area 11 that can be effectively attached to the skin of a wearer on either side of the labia majora.

In addition, the article 10 may also be configured to have an anterior portion 64, a central portion 65 and a posterior portion 66, as is shown in FIG. 9. As used herein, the term “anterior” refers to the direction towards the front of the wearer during use. As used herein, the term “posterior” refers to the direction towards the back of the wearer during use. A particular aspect is shown in FIG. 9 of an article having a configuration designed to fit specific areas of the vulva region of a wearer. By providing specific portions for attachment to specific areas of the body of the wearer, the article may be configured to better fit the body of the wearer. The anterior portion 64 of the article in the illustrated embodiment will be the portion of the article between the absorbent structure 21 and the first end 61 of the article 10. The posterior portion 66 of the article 10 in the illustrated embodiment will be the portion of the article between the absorbent structure 21 and the second end 62 of the article 10. (In the illustrated embodiment of FIG. 9, the portions are discerned by dashed line 68). Generally, the posterior portion 66 will be designed to be placed between the vagina area and the anal area of the wearer. The anterior portion 64 is designed to be placed on the mons veneris region of a female wearer. The central portion 65 of the article 10 is designed to cover the vagina area of the wearer and the skin area surrounding the lateral sides of the labia majora, when the article is used as a pantiliner, sanitary napkin or an incontinence article. In an alternative use, the article of the present invention may also be used as an underwear replacement, or a guard for a swimming suit.

To obtain an effective attachment to the body of the wearer, the shell 14 can be configured to be anatomically correct for a wearer. As is shown in FIGS. 9, 12 and 20A-C, the shape of the article 10 is such that it will correctly and securely fit in the vulva region of a wearer. The general shape of the article has been found to effectively attach to the vulva region of female wearers of the article. Additional features may be included to ensure an anatomically correct shape. For example, in the posterior region of the article 10 of FIGS. 9 and 10, more particularly, the posterior region of the shell on the first side 15, the shell 14 may be imparted with a three-dimensional protrusion 67. The protrusion 67 acts to fit comfortably in the perinea region of the wearer. The protrusion 67 may be formed from the shell material or may be formed from the body adhesive 44. By providing the three-dimensional protrusion 67, the article can effectively fit to the typical body shape of the female wearer, thereby preventing leaks form the posterior portion of the article. The protrusion 67 may also serve as a guide to the wearer in placement of the article 10 on the body prior to use.

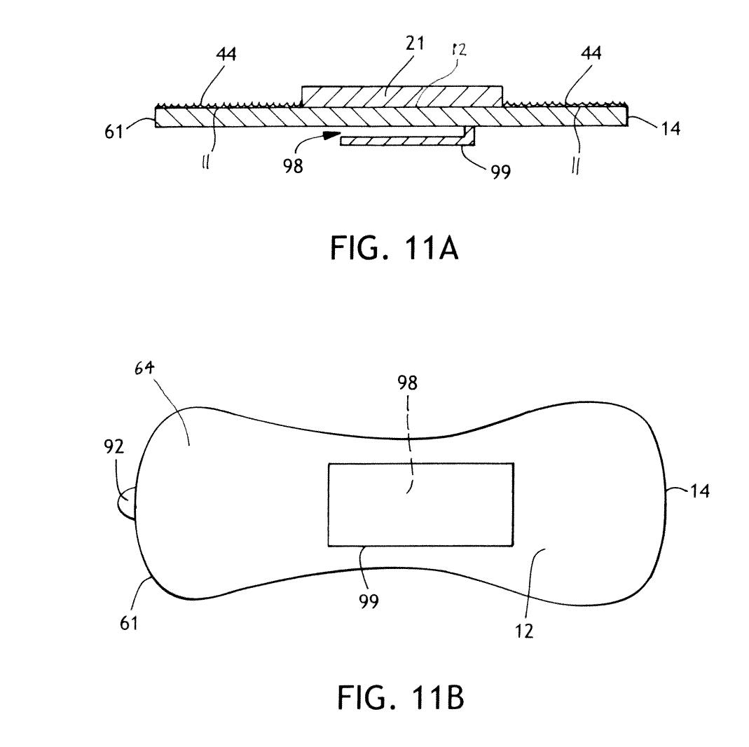

The article of the present invention may have other features which aid the wearer to place and remove the article from the body. As is shown in FIGS. 11A and 11B, the second side 12 of the shell 14 may be provided with positioning aids such as a finger pocket 99 and/or finger grooves in the shell material. The finger pocket 99 has an opening 98 towards the anterior portion 64 or first end 61 of the article 10. The pocket 99 gives the wearer a location to place her fingers during placement of the article 10 onto the wearer's body. The pocket 99 may be an opening wide enough for the wearer to place one or more fingers in the pocket. Alternatively, the article can comprise two or more openings which allows the wearer to place only one finger in each opening. Other similar positioning aids may be used to help guide a wearer to properly position the article for use. For example, grooves may be placed in the second side 12 of the shell 14 opposite the absorbent structure 21. This can allow the wearer to feel the location of the absorbent structure 21 relative to the vulva region during application of the article 10 to the vulva region of the body. The pocket 99 can also assist the wearer in removing the article from their body.

In some aspects, the article 10 can also be provided with a removal aid which provides the wearer with an easy way to grasp and remove the article when attached to the body. One particular removal aid is shown in FIG. 11B including a tab 92 located on the first end 61 of the shell which is not adhered to the body or is devoid of adhesive. Alternatively, other removal aids, such as having an area of the first end 61 being devoid of the body attaching adhesive 44. Other types of removal aid which may be present include loops, pull strings and the like. The removal aid allows the wearer to effectively begin the process of gently removing the article from the body of the wearer, without the need of having to find a portion of the shell which may not be completely attached.

Other features or additives may be incorporated into the article of the present invention. For example, the article may contain an odor control agent, a fragrance, a skin wellness agent and other similar additives used in currently available absorbent articles. Any odor control agent, fragrance or wellness agent known to those skilled in the art can be used in the article of the present invention. The odor control agent, fragrance or wellness agent may be added in various components of the article, including the shell 14, the absorbent structure 21 or the body adhesive 44, for example.

Generally, to apply the article 10 to the body of a wearer, the release sheet 46 protecting the absorbent structure and adhesive, if present, is removed from the first surface 15 of the shell 14. Next, the wearer positions the shell 14, or absorbent structure 21, if present, onto the portion of the body in which protection is desired. If positioning pockets or other positioning aids are present on the article, the wearer may optionally use these positioning aids to properly place the article for use. In the case of sanitary napkins and incontinence absorbent articles for females, the absorbent structure 21 is positioned over the vagina area such that the absorbent structure will absorb body fluids. The wearer then checks to ensure that the first area 11 of the shell or the adhesive 44 is contacting the skin around the vagina area.

If the article is intended to have a front and a back portion, the wearer first identifies the anterior portion 64 and/or the posterior portion 66 of the article. To aid in identification of the anterior and posterior portions, indicia located on the release sheet, shell or absorbent structure to indicate the anterior portion and/or posterior portion of the article may be present. Indicia can be simply lettering or a picture to indicate the front or back of the article. Once the anterior portion and posterior portion are identified by the wearer, the wearer places the article in the same manner described above. Examples of indicia which may be used include, color, wording, diagrams and the like, which would indicate to a wearer the anterior and posterior regions of the article. In some aspects, a set of instructions can also be included with the article which aid the wearer in determining proper placement and use.

In aspects comprising an absorbent structure, which can be designed to cover the labia majora of the wearer, the article can be positioned with the aid of the absorbent structure. More specifically, the absorbent structure, when sized and shaped to the approximate size of the labia majora, can serve to guide the placement of the absorbent structure over the labia majora. Once properly placed, pressure is applied by the wearer to the second surface of the shell which will allow the first surface of the shell to contact the skin of the wearer, or to allow any adhesive applied to the first surface to be applied to the skin of the wearer.

By having the article 10 attached to the body of a wearer, the article 10 will desirably move with the skin of the wearer. This results in an article that is comfortable to wear and which will be less likely to leak than conventional personal care articles. The article of the present invention advantageously has a very close to the body fit which can provide improved discretion and confidence for the wearer as compared to conventional personal care articles.

Other benefits of the article 10 of the present invention may also be provided. For example, when the first side of the shell has an adhesive applied thereto, upon removal of the article after use, the wearer may fold the first side of the shell onto itself to dispose of the used article. An effective seal may be formed around the perimeter of the shell, thereby effectively encapsulating bodily fluids within a closure. As a result, any odors associated with the fluids will be contained within the shell material. Another advantage of the article of the present invention is a tampon backup article. The article can be effective in hiding the withdraw string of a tampon, while providing additional leakage protection.

The article described above can be an individual article or may be part of a personal care system, offering the wearer a wide variety of options to fill the needs of the wearer. For example, the shell can be provided to wearers in a variety of shapes or sizes to allow wearers to select the appropriate shape or size for their given body shape or degree of fluid flow. Likewise, the body adhesive may be provided in a variety of adhesive strengths to match the adhesive strength needed or desired by the wearer. By providing a variety of adhesive or other attachment means, a wearer could select the shells to match body type, body condition and other various factors that may vary from one wearer to another. Similarly, the absorbent structure could be provided in various absorbent capacities so that the wearer could select the appropriate absorbency to match the wearer's needs.

The personal care system may be provided to wearers in a variety of packaging arrangements. In one packaging arrangement, a plurality of shells having different properties may be provided in separate packages or could be provided in a single package. It is generally a better packaging arrangement if shells having similar properties, shapes or sizes are provided in a single package. That is, in a given package, the wearer is provided with a plurality of shells all having the same shape, size, and properties, such as similar body attachment properties. Regarding the absorbent structures, the absorbent structures could be provided to the wearer in packages sorted by absorbent capacity or various absorbent capacity structures could be provided in a single package. By having all absorbent structures in a single package with a single absorbent capacity, a wearer is able to select the correct absorbent capacity for their typical needs. However, by providing different absorbent capacity absorbent structures in a single package, the wearer will be provided with the ability to select the absorbent structure with the appropriate absorbent capacity for a given situation, without the need to purchase multiple packages of absorbent structures.

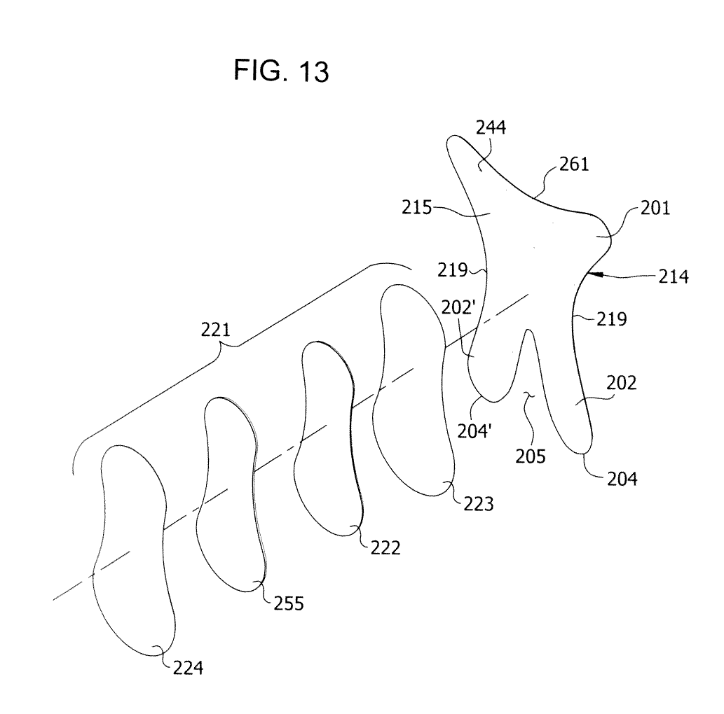

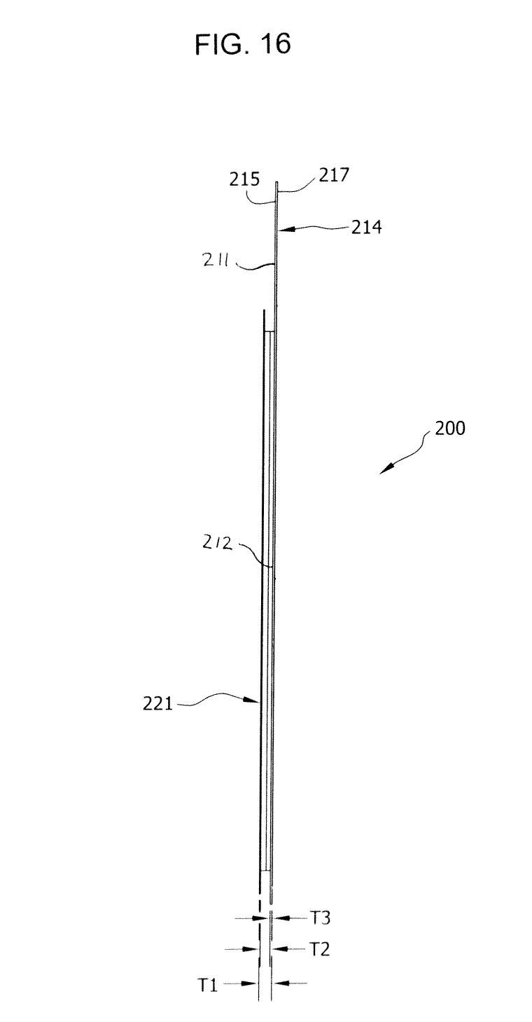

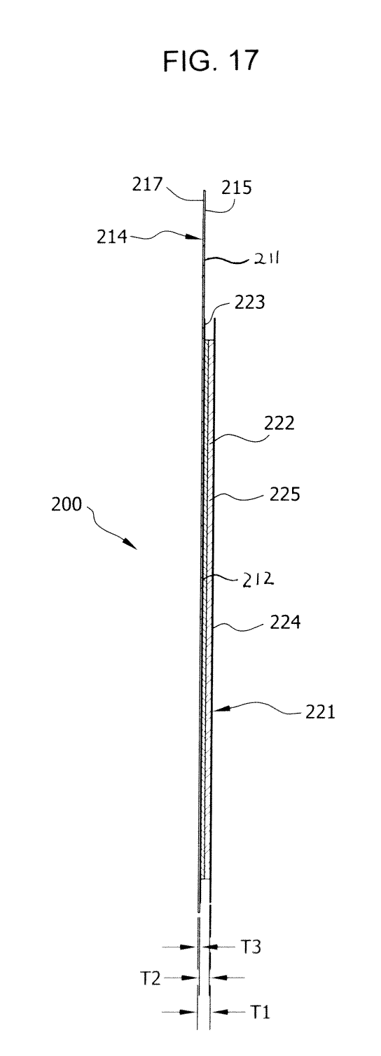

In another embodiment, a body adhesive personal care article 200, which is illustrated in FIGS. 12-19, comprises a shell 214 and an absorbent structure 221 and has a longitudinal axis 1 and a transverse axis 2. The shell 214 has a first region 201, a pair of lateral side regions 202, 202′ extending from the first region, and an opening 205 (FIG. 13) extending longitudinally at least in part between the side regions. The shell 214 also has a first side 215, which defines a body-facing surface (FIG. 12), and a second side 217, which defines a garment-facing surface (FIG. 15). The first side 215 of the shell 214 also has first area 211 and a second area 212. In the illustrated embodiment, the first side 215 of the shell 214 has a body adhesive 244 on at least a portion thereof for adhering the article 200 directly to the wearer's skin, and particularly, to a female wearer's skin surrounding her vulva region for the illustrated article. The body adhesive 244 contacts the skin and hair, if present, in the vulva region and possibly the pubic region and/or the perinea region of the wearer's body, thereby supporting and holding the shell 214 and absorbent structure 221 against the body of the wearer during use. A peel sheet or release sheet (not shown) may be used to prevent the body adhesive 244 from becoming contaminated, thus losing its ability to stick to the body of the wearer and/or prematurely adhering to an unintended surface.

Generally, the size and shape of the absorbent structure 221, depending on its intended use, will dictate the size of the shell 214. The shape of the shell 214 is selected so that the article 200 will have a comfortable feeling for the wearer and inhibit the article against becoming detached from the body of the wearer during use thereby providing protection against leaks. In one aspect, the article 200, including the shell 214 and absorbent structure 221, is dimensioned and shaped to fit approximately 75 percent of adult females. It is understood, however, that the article 200 can be dimensioned and shaped to fit more or fewer females. It is also contemplated that different sizes of the article 200 may be provided to accommodate a greater percentage of females.

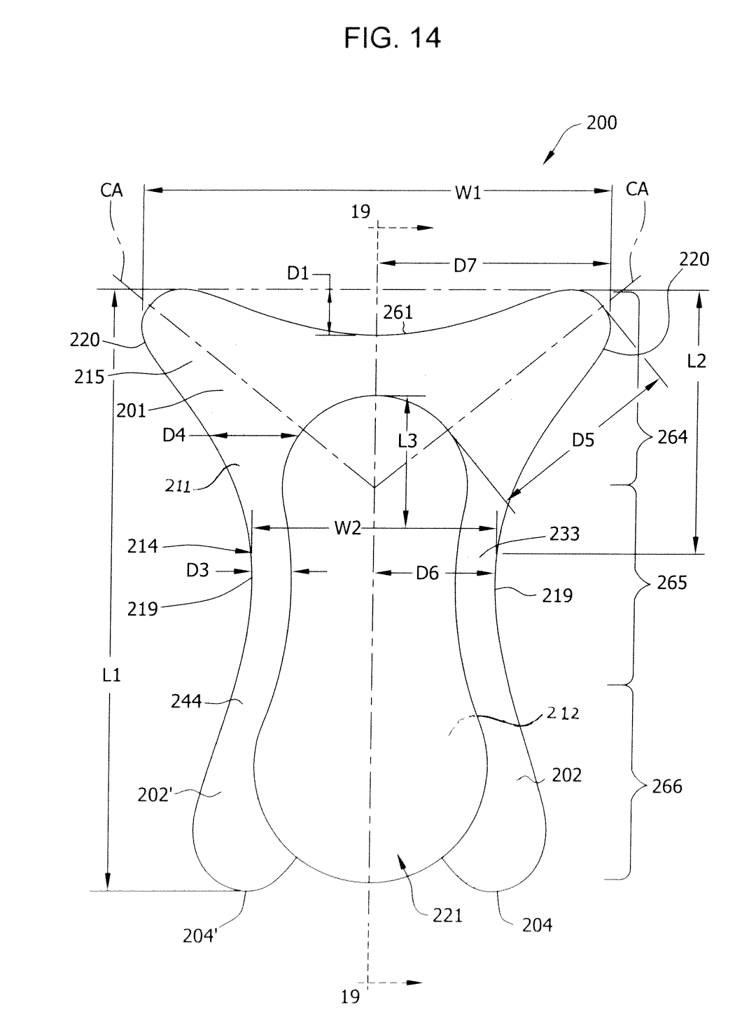

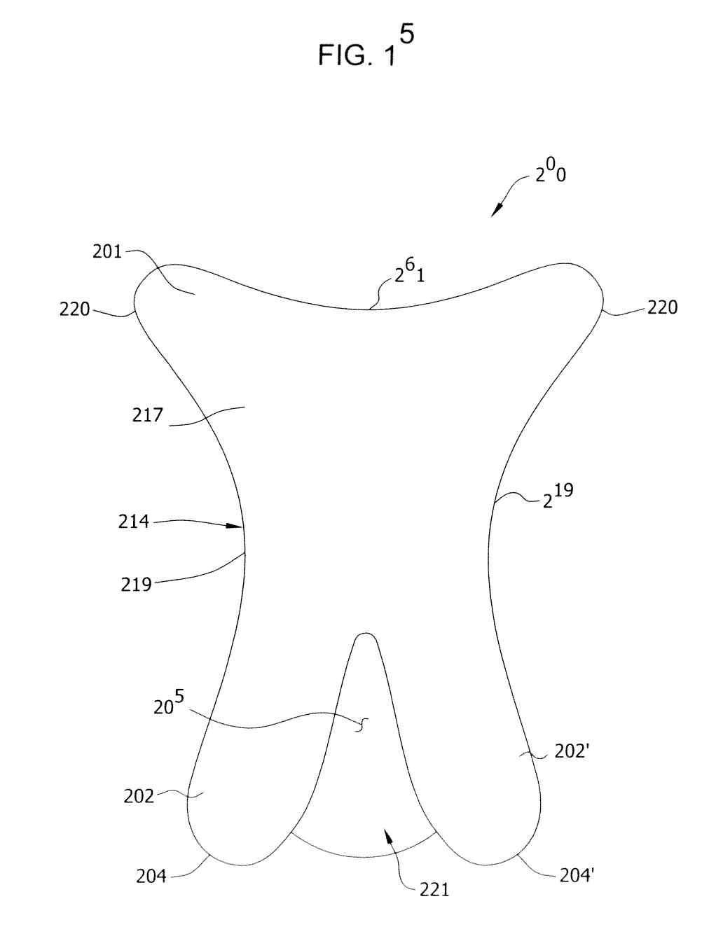

With reference to FIG. 14, the article 200 (and hence the shell 214) can be suitably divided into three general longitudinal regions: an anterior region 264, a posterior region 266 and a central region 265 extending longitudinally between and interconnecting the anterior and posterior regions. Each of these regions 264, 265, 266 is sized and shaped for alignment with different body regions of a wearer of the article. More specifically, the anterior region 264 of the article 200 is adapted to be disposed adjacent the wearer's lower abdomen region. The central region 265 is adapted to be disposed between the upper thigh region of the wearer to cover the wearer's perineum region and vaginal region. The posterior region 266 of the article 200 is adapted to be disposed in the gluteal region of the wearer.

In the illustrated embodiment, the anterior region 264, the central region 265, and the posterior region 266 of the article 200 are of roughly equal length, with each region corresponding generally to about ⅓ of a total length L1 of the article 200. The length L1 is defined herein as the longitudinal distance from a longitudinally outermost extent of the article 200 (and in the illustrated embodiment, the shell 214) in the anterior region 264 to a longitudinally outermost extent of the article (and in the illustrated embodiment, the shell) in the posterior region 266. As an example, the length L1 of the shell 214 (and hence the article 200 in the illustrated embodiment) may suitably be in the range of about 170 mm to about 220 mm, and more suitably in the range of about 190 mm to about 200 mm. As an additional example, the article 200, and more particularly the shell 214, has a length L1 of about 194 mm. It is understood that the article 200 may have a length L1 different than those set forth above without departing from some aspects of this invention. It is also contemplated that two or all three of the article regions 264, 265, 266 may instead be of unequal lengths depending on the desired fit and the intended body placement of the article without departing from the scope of this invention.

The absorbent structure 221 of FIGS. 14-19 is suitably adhered to the first side (i.e., body-facing surface) 215 of the shell 214 and is sized and located relative to the shell such that the shell extends both longitudinally and transversely outward beyond the periphery of the absorbent structure in at least the anterior region 264 and the central region 265, and more suitably in at least a portion of the posterior region 266 as well. The absorbent structure 221 is offset longitudinally, i.e., not centered lengthwise on the transverse or lateral axis of the article, such that the shell 214 extends longitudinally outward beyond the absorbent structure a greater distance in the anterior region 264 of the article 200 than in the posterior region. It is understood, though, that the absorbent structure 221 may be longitudinally centered so that the shell 214 extends equally longitudinally outward beyond the absorbent structure, or may be offset longitudinally toward the anterior region 264 so that the outward longitudinal extension of the shell beyond the absorbent structure is greater in the posterior region 266 than in the anterior region without departing from the scope of this invention.

As illustrated in FIG. 14, the anterior region 264 of the article 200 comprises the first region 201 of the shell 214 and includes a portion of the absorbent structure 221. Since much of the first side (i.e., body-facing surface) 215 of the shell 214 is exposed (i.e., not covered by the absorbent structure 221) in the anterior region 264 of the article 200, a relatively large surface area of the first side of the shell has body adhesive 244 applied thereto for adhering the shell, and hence the article, to the wearer.

A first end 261 of the article 200, and more particularly a longitudinal edge of the anterior region 264 defining this first end of the article 200, is suitably contoured along the width of the shell at this first end to accommodate the lower abdomen region of the wearer. In the illustrated embodiment, for example, the longitudinal extent (e.g., length) of the shell 214 relative to the transverse axis of the article is non-uniform across the width of the shell at the first end 261 of the article, and more suitably increases as the shell extends transversely outward from the longitudinal axis of the article to transversely, or laterally opposite sides 219 of the article and more particularly laterally opposite side edges of the shell. Accordingly, a greatest longitudinal extent of the shell 214 is generally adjacent the intersection of the longitudinal end 261 with the respective sides 219 of the article (i.e., the shell in the embodiment of FIG. 14). More suitably, the longitudinal edge of the shell 214 (i.e., at first end 261 of article 200 in the illustrated embodiment) is generally inwardly arcuate as it extends across the width of the shell at its longitudinal edge. It is understood, however, that the contour of the longitudinal edge of the shell 214 in the anterior region 264 of the article may be V-shaped, U-shaped, transversely straight, outwardly arcuate or other suitable shape without departing from the scope of this invention.

The contoured longitudinal edge of the shell 214 (i.e., first end 261 of the article 200 in the illustrated embodiment) thus broadly defines a recess in the anterior region 264 of the article (and thus of the shell in this instance). This recess defines a longitudinal distance D1 between the longitudinally outermost extent of the longitudinal edge of the shell 214 in the anterior region 264 and the longitudinal extent of the longitudinal edge of the shell at the longitudinal axis of the article 200 in the anterior region. In one suitable embodiment, the distance D1 of the recess is in the range of about 5 mm to about 35 mm, and more suitably about 12 mm to about 18 mm. As one example, the distance D1 of the recess at the anterior region 264 in the embodiment of FIG. 14 is approximately 15 mm.

The sides 219 of the illustrated article 200 are suitably defined by transversely opposite side edges of the shell 214. These side edges of the shell 214 are contoured so that the overall width of the article 200 (i.e., the distance between the transversely opposite sides 219 thereof), and more particularly the width of the shell in the illustrated embodiment, is non-uniform along the length L1 of the article to define leg cutouts for accommodating the upper thighs of the wearer. In one aspect, the width of the article 200 and hence the shell 214 increases from a narrowest width W2 in the central region 265 of the article toward each of the longitudinally opposite ends (261 and 204, 204′) of the article. More suitably, the width of the article 200 and more suitably the shell 214 is also greater in the anterior region 264 of the article than in the posterior region 266. In the illustrated embodiment, for example, a greatest width W1 of the article 200 is defined by the transverse side edges of the shell 214 adjacent the longitudinal edge of the shell (e.g., first end 261 of the article 200) in the anterior region 264 of the article. As additional examples, the greatest width W1 of the article 200 and more particularly the shell 214 is in the range of about 52 mm to about 180 mm and more suitably about 140 mm to about 170 mm. In the illustrated embodiment of FIG. 14, the greatest width W1 of the article 200 is approximately 150 mm. The narrowest width W2 of the article 200 and more particularly the shell 214 is in the range of about 45 mm to about 85 mm, and more suitably about 60 mm to about 80 mm. In the illustrated embodiment, for example, the narrowest width W2 of the shell 214 is approximately 78 mm. In other embodiments, a ratio of the length L1 of the shell 214 (and hence the article 200 in the illustrated embodiment) to the narrowest width W2 of the shell 214 (and hence article 200) is in the range of about 3 to about 1, and more suitably about 2 to about 1.

In the article 200 illustrated in FIG. 14, the sides 219 of the article 200 and more particularly the transverse side edges of the shell 214 are generally inwardly arcuate along substantially the entire length L1 of the article. Alternatively, the sides 219 may be inwardly arcuate along only a portion of the length L1 of the article. It is also understood that the sides 219 defining the leg cutouts may be V-shaped, U-shaped, outwardly arcuate or other suitable shape, or it they may be uniform (e.g., straight or longitudinal) along substantially the entire length L1 of the article 200. It is also understood that the sides 219 of the article may be contoured to define article 200 widths other than those set forth above without departing from the scope of this invention. It is further understood that the greatest width of the article 200 may be other than in the anterior region 264, and/or the narrowest width may be other than in the central region 265 of the article and remain within the scope of this invention.

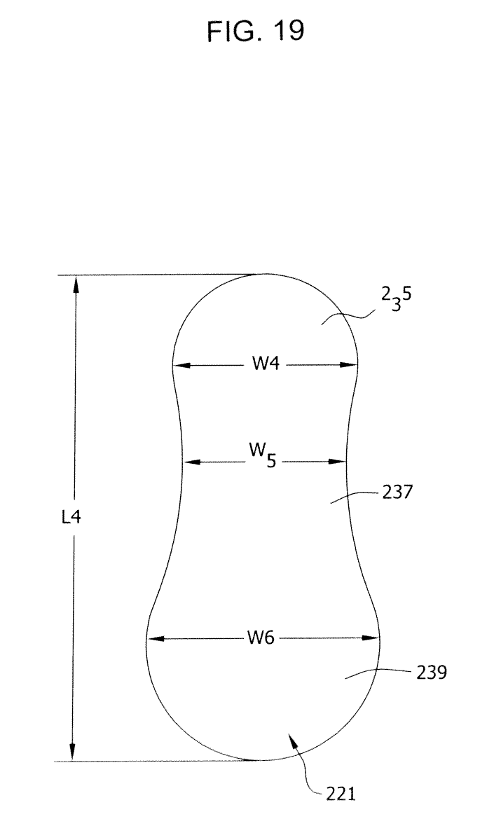

Still referring to FIG. 14, the contoured longitudinal edge of the shell 214 (e.g., first end 261 of the article 200) at the anterior region 264, together with the contoured transverse side edges of the shell (e.g., article sides 219) where these side edges generally intersect the longitudinal edge of the shell, define a pair of transversely spaced tabs 220 in the anterior region. Each tab 220 has a central axis CA extending in part transversely outward of the shell 214 and in part longitudinally outward of the shell. Each of the tabs 220 suitably has body adhesive 244 on the body-facing surface (e.g., first side 215) for adhering the tabs directly to the wearer and more suitably to the abdomen region of the wearer. In one particular aspect, the tabs 220 are sized to extend to a region of the wearer that has little or no pubic hair to facilitate better adherence to the wearer's skin. For example, in one aspect each of the tabs 220 extends outward along its central axis CA away from the peripheral edge of the absorbent structure 221 a distance D5 in the range of about 20 mm to about 90 mm, and more suitably about 45 mm to about 70 mm. Each tab 220 also has a transversely outermost extent (which in the illustrated embodiment defines the greatest width W1 of the shell 214 and hence the article 200) defining a distance D6 from the longitudinal axis of the article to the transversely outermost extent of a respective one of the tabs (which is approximately half of the width W1 of the shell). In a particularly suitable embodiment, a ratio of the distance D6 (that the tab 220 extends transversely outward) to the distance D5 (the length of the tab along its central axis CA) is in the range of about 1 to about 2. In another suitable embodiment, a ratio of the distance D6 to a distance between the longitudinal axis of the shell 214 and a side edge of the absorbent structure 221 (i.e., about half of the width W5 shown in FIG. 19) is in the range of about 2 to about 5. However, in some aspects, it is desirable to provide a shape that attaches to the hairs in the region.

Each of the tabs 220 further has a longitudinally outermost extent (which in the illustrated embodiment defines the outermost extent of the longitudinal edge of the shell 214) in the anterior region 264 defining a length L2 from the transverse axis of the shell 214 to the longitudinally outermost extent of the tab 220. This length L2 is suitably in the range of about 50 mm to about 120 mm, and more suitably about 70 mm to about 100 mm. As illustrated in FIG. 14, the absorbent structure 221 extends longitudinally into the anterior region 264 of the article and has a longitudinally outermost extent defining a length L3 from the transverse axis to the longitudinally outermost extent of the absorbent structure in the anterior region. For example, this length L3 may suitably be in the range of about 30 mm to about 90 mm, and more suitably about 50 mm to about 70 mm. In another embodiment, a ratio of the length L2 (the longitudinally outermost extent of the tabs 220) to the length L3 (the longitudinally outermost extent of the absorbent structure 221 in the anterior region 264) is in the range of about 3 to about 1 and more suitably about 2 to about 1.

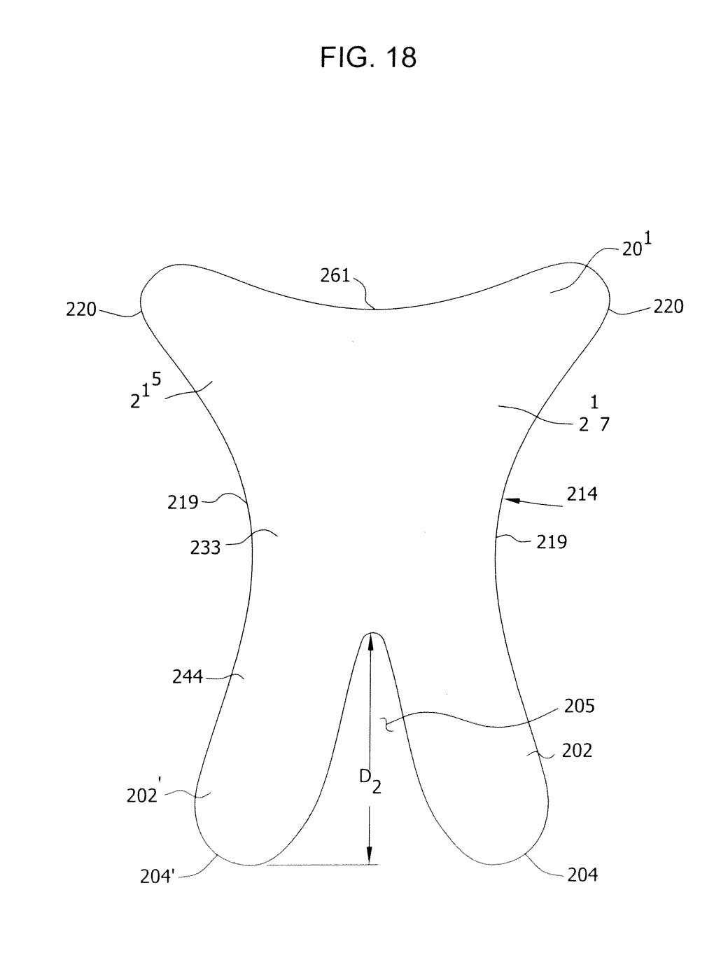

With reference now to FIGS. 14 and 18, the posterior region 266 of the article 200 includes the opening 205 in the shell 214 with portions of the lateral side regions 202, 202′ broadly defining a pair of transversely spaced tabs disposed on opposite sides of the opening. The posterior region 266 disposition of these tabs is such that the tabs are aligned generally with the buttocks of the wearer rearward of the perineal region. In the illustrated embodiment, the opening 205 is in the form of a generally V-shaped ingress extending longitudinally on the longitudinal axis of the article 200 such that the tabs are free to flex relative to the central region 265 of the article and generally independent of each other to accommodate normal movement of the wearer's thighs and buttocks. In one particular aspect, the ingress 205 extends longitudinally inward from the distal end 204, 204′ of the article 200 (and more particularly a greatest longitudinal extent of the shell in the posterior region 266) a distance D2 in the range of about 5 mm to about 100 mm, and more suitably about 50 mm to about 80 mm. In the illustrated embodiment, for example, the ingress 205 has a distance D2 of about 75 mm. In another embodiment, the distance D2 of the ingress 205 is in the range of about 5 percent to about 60 percent of the length L1 of the shell 214, and more suitably about 25 percent to about 40 percent of the length L1. In other aspects, a ratio of the distance D1 of the recess in the anterior region 264 of the shell 214 to the distance D2 of the ingress 205 in the posterior region 266 is in the range of about 4 to about 1, and more suitably between about 3 and about 1. In still other aspects, a ratio of the distance D1 of the recess in the anterior region 264 of the shell 214 to the total length L1 of the shell is suitably in the range of about 0.03 to about 0.2 and more suitably in the range of about 0.06 to about 0.09. It is understood, however, that the ingress 205 can be larger or smaller without departing from some aspects of this invention.

Turning now to FIGS. 13, 16 and 17, the absorbent structure 221 can comprise a single layer structure or be constructed of multiple layers. The illustrated absorbent structure 221, for example, comprises an absorbent core 222, an intake layer 225, a topsheet 224, and a liquid impermeable backsheet 223. A total thickness T1 of the article 200 is suitably in the range of about 1 mm to about 12 mm, and more suitably about 2.5 mm to about 5 mm. As one example, the thickness T1 of the illustrated article is approximately 3.5 mm. It is understood, however, that the thickness T1 may be other than as set forth above depending at least in part on the intended use of the article 200. For example, an article 200 in which the absorbent structure 221 is intended to be used in the manner of a maxi-pad may have a greater thickness T1 than an article in which the absorbent structure is to be used in the manner of a panty-liner. In another aspect, the absorbent structure 221 has a thickness T2 in the range of about 1 mm to about 12 mm, and more suitably in the range of about 1.5 mm to about 5 mm. In the illustrated embodiment, for example, the thickness T2 of the absorbent structure is approximately 3 mm. The shell 214 itself may have a thickness T3 between about 0.03 mm and about 5.0 mm, and more suitably about 0.1 mm to about 3.0 mm. In one particularly suitable embodiment, the thickness T3 of the shell 214 is between 0.25 mm and about 3.0 mm. In the illustrated embodiment, for example, the shell 214 has a thickness T3 of about 0.5 mm.

With reference now to FIG. 19, the illustrated absorbent structure 221 has an upper portion 235, a middle portion 237, and a lower portion 239. The absorbent structure is generally hourglass shaped, with the upper portion 235 suitably having a width W4 between about 10 mm and about 80 mm, and more suitably about 30 mm to about 60 mm. In the illustrated embodiment, for example, the width W4 of the upper portion 235 is approximately 47 mm. The middle portion 237, which is the narrowest portion of the absorbent structure 221, may have a width W5 between about 10 mm and about 80 mm, and more suitably about 30 mm to about 60 mm. In the illustrated embodiment, the width W5 of the middle portion 237 is approximately 40 mm. The lower portion 239 has a width W6 between about 10 mm and about 120 mm, and more suitably about 40 mm to about 80 mm. In the illustrated embodiment, for example, the width W6 of the lower portion 239 is approximately 63 mm. In another aspect, the absorbent structure 221 has a longitudinal length L4 in the range of about 80 mm to about 180 mm, and more suitably about 110 mm to about 150 mm. As one example, the longitudinal length L4 of the illustrated absorbent structure 221 is about 145 mm. It is understood, however, that the absorbent structure may be sized in width and/or length other than as set forth above without departing from the scope of this invention. It is also understood that the absorbent structure 221 may be any suitable shape other than a generally hourglass shape within the scope of this invention.

With reference back to FIG. 14, the absorbent structure 221 is secured to the first side (i.e., body-facing surface) 215 of the shell 214, such that at least a portion of the absorbent structure covers the opening or ingress 205 in the shell. The absorbent structure 221 may be attached to the shell 214 in a permanent manner, meaning that the absorbent structure is generally intended not to be removable by the wearer of the article 200. Alternatively, it may be removably and in some embodiments refastenably) attached to the shell 214, such that the absorbent structure 221 may be removed (and in some embodiments reattached) by a wearer.

The shell 214 and absorbent structure 221 are sized relative to each other such that a portion of the shell extends outward beyond the peripheral edge of the absorbent structure along at least a portion of the peripheral edge of the absorbent structure. In this manner, a portion of the shell 214 about the periphery of the absorbent structure 221 is uncovered with the first side (i.e., body-facing surface) 215 of the shell exposed and available for adhesion to the wearer. For example, the shell 214 in one suitable embodiment extends outward beyond the peripheral edge of the absorbent structure 221 at least in the anterior region 264 and central region 265, and more suitably also in a portion of the posterior region 266. In accordance with one embodiment, for example, the shell 214 extends outward of the peripheral edge of the absorbent structure 221 a distance D3 in the range of at least about 3 mm, more suitably in the range of about 5 mm to about 15 mm and even more suitably about 8 mm to about 13 mm. In one aspect, the entire first side 215 of the uncovered portion of the shell 214 has body adhesive 244 thereon for adhering the shell and thereby the article to the wearer.

As illustrated in FIG. 14, the distance that the shell 214 extends outward beyond the peripheral edge of the absorbent structure 221 is suitably non-uniform about the periphery of the absorbent structure. More particularly, the shell 214 extends transversely outward beyond each of the side edges of the absorbent structure 221 a greater distance in the anterior region 264 than in the central region 265. It is understood, however, that shell 214 may extend a uniform distance outward of the absorbent structure 221, or may extend outward according to a different pattern than illustrated in FIG. 14, and remain within the scope of this invention. In another aspect, the first side (i.e., body-facing surface) 215 of the shell 214 has a total surface area in the range of about 50,000 mm² to about 20,000 mm², and more suitably about 30,000 mm² to about 40,000 mm². The absorbent structure 221 has a total body-facing surface area of about 4,500 mm² to 45,000 mm² and more suitably about 15,000 mm² to about 20,000 mm². Thus, between about 10,000 mm² and about 45,000 mm², and more suitably about 18,000 mm² to about 22,000 mm² of surface area of the first side 215 of the shell 214 remains uncovered by the absorbent structure 221. Stated another way, about 40 percent to about 95 percent, and more suitably about 40 percent to about 65 percent of the shell 214 is uncovered by the absorbent structure 221.

As one example, in the illustrated embodiment the shell 214 has a total surface area of about 34,000 mm² of which about 20,000 mm² is uncovered and available to have body adhesive 244 applied thereto. The illustrated absorbent structure 221 has a total body-facing surface area of about 18,000 mm² of which about 14,500 mm² covers or overlies the shell 214. Accordingly, about 60 percent of the illustrated shell 214 has body adhesive 244 and can be used to adhere the article 200 to the wearer's skin. It is understood, however, that less than the entire exposed area of the shell 214 can have body adhesive 244 thereon. It is also understood that body adhesive can be applied to the absorbent structure 221 to adhere or partially adhere the absorbent structure to the wearer's skin.

FIG. 20A illustrates another embodiment of a body adhesive personal care article 300 comprising a shell 314 and an absorbent structure 321. The shell 314 has a first region 301 and an elongate region 302 extending from the first region. The shell 314 also has a first side 315, which defines a body-facing surface (FIG. 20A), and a second side, which defines a garment-facing surface (317). The first side 315 of the shell 314 also has first area 311 and a second area 312. (In the illustrated embodiment of FIG. 21A, the first area and second area are discerned by dashed line 378). In the illustrated embodiment, the first side 315 of the shell 314 has a body adhesive 344 on at least a portion thereof for adhering the article 300 directly to the wearer's skin, and particularly, to a female wearer's skin surrounding her vulva region for the illustrated article. The body adhesive 344 contacts the skin and hair, if present, in the vulva region and possibly the pubic region and/or the perinea region of the wearer's body, thereby supporting and holding the shell 314 and the absorbent structure 321 against the body of the wearer during use.

In the illustrated embodiment, the shell 314 contains adhesive 344 in only a portion of the first region 301 and in only a portion of the elongate region. It is understood that the entire exposed area (i.e., the area not covered by the absorbent structure 321) or other portions of the exposed area of the first side 315 of the shell 314 can have adhesive 344 thereon without departing from the scope of this invention. A releasable peel sheet or release sheet (not shown in FIG. 20A) may be used to prevent the body adhesive 344 from becoming contaminated, thus losing its ability to stick to the body of the wearer and/or prematurely adhering to an unintended surface. The absorbent structure 321 is suitably secured to the first side (i.e., body-facing surface) 315 of the shell 314 and is sized and located relative to the shell such that the shell extends longitudinally outward beyond the periphery of the absorbent structure. In some aspects, the article can include an absorbent material 327 coated onto and/or integrated into the shell 314.

A first end 361 of the article 300 is suitably contoured along the width of the shell at the first end to accommodate the lower abdomen region of the wearer. In the illustrated embodiment, for example, the longitudinal extent (e.g., length) of the shell 314 relative to the transverse axis of the article is non-uniform across the width of the shell at the first end 361 of the article 300, and more suitably increases as the shell extends transversely outward from the longitudinal axis of the article to transversely, or laterally opposite sides 319 of the article and more particularly laterally opposite side edges of the shell. Accordingly, a greatest longitudinal extent of the shell 314 is generally adjacent the intersection of the longitudinal end 361 with the respective sides 319 of the article (i.e., the shell in the embodiment of FIG. 20A). More suitably, the longitudinal edge of the shell 314 (i.e., at first end 361 of article 300 in the illustrated embodiment) is generally V-shaped as it extends across the width of the shell at its longitudinal edge. It is understood, however, that the contour of the longitudinal edge of the shell 314 may be inwardly arcuate, U-shaped, transversely straight, outwardly arcuate or other suitable shape without departing from the scope of this invention. For example, FIG. 20B shows an article where the longitudinal edge of the shell 314 is transversely straight at the longitudinal end 361, and FIG. 20C shows an article where the longitudinal edge of the shell 314 is outwardly arcuate at the longitudinal end 361.

In the article 300 illustrated in FIG. 20A, the sides 319 of the article 300 and more particularly the transverse side edges of the shell 314 are generally inwardly arcuate along substantially the entire length of the article. Alternatively, the sides 319 may be inwardly arcuate along only a portion of the length of the article. It is also understood that the sides 319 defining the leg cutouts may be V-shaped, U-shaped, outwardly arcuate or other suitable shape, or it may be uniform (e.g., straight or longitudinal) along substantially the entire length of the article 300.

The contoured longitudinal edge of the shell 314 (e.g., first end 361 of the article 300) together with the contoured transverse side edges of the shell (e.g., article sides 319) where these side edges generally intersect the longitudinal edge of the shell, define a pair of transversely spaced tabs 320. Each of the tabs 320 suitably has body adhesive 344 on the body-facing surface (e.g., first side 315) for adhering the tabs directly to the wearer and more suitably to the abdomen region of the wearer. In one particularly suitable embodiment, the tabs 320 are sized to extend to a region of the wearer that has little or no pubic hair to facilitate better adherence to the wearer's skin. However, in some aspects, it is desirable to provide a shape that attaches to the hairs in the region.

The elongate portion 302 of the shell 314 includes a tab 302′ for generally aligning with the coccyx of the wearer. The tab 302′ has adhesive thereon for adhering the shell 314 to the body of the wearer. The tab 302′ of the illustrated embodiment is generally bulbous in shape but it is understood that the tab could have other shapes. In some aspects, the elongated portion can rely on friction within the gluteal groove to provide additional attachment of the article to the body. In further embodiments of this aspect, the elongated portion 302 comprises no body adhesive.

It will be appreciated that details of the foregoing examples, given for purposes of illustration, are not to be construed as limiting the scope of this invention. Although only a few exemplary embodiments of this invention have been described in detail above, those skilled in the art will readily appreciate that many modifications are possible in the examples without materially departing from the novel teachings and advantages of this invention. For example, features described in relation to one example may be incorporated into any other example of the invention.

Accordingly, all such modifications are intended to be included within the scope of this invention, which is defined in the following claims and all equivalents thereto. Further, it is recognized that many embodiments may be conceived that do not achieve all of the advantages of some embodiments, particularly of the desirable embodiments, yet the absence of a particular advantage shall not be construed to necessarily mean that such an embodiment is outside the scope of the present invention. As various changes could be made in the above constructions without departing from the scope of the invention, it is intended that all matter contained in the above description shall be interpreted as illustrative and not in a limiting sense.

1. A personal care article comprising:
an absorbent structure configured for disposition adjacent a wearer's vulva region; and
a shell for supporting the absorbent structure at the vulva region;
wherein the shell has a first side and a second side, the first side having a first area and a second area;
wherein the second area of the shell has a second area circumference, and the first area of the shell surrounds and bounds at least 1% of the second area circumference;
wherein at least a portion of the first area comprises a body adhesive for adhering the shell directly to the wearer;
wherein the absorbent structure is present in the second area of the shell; and
wherein the first area of the shell contacts to the wearer's skin and/or hair surrounding the vulva region.

2. The personal care article according to claim 1, wherein the first area of the shell surrounds and bounds between 25%-100% of the second area circumference.

3. The personal care article according to claim 1, wherein the shell comprises a woven web, nonwoven web, a gel, a film, a sheet of a polymeric material, a foam, or a laminate.

4. The personal care article according to claim 1, wherein the shell comprises a silicone material.

5. The personal care article according to claim 1, wherein the first area of the shell comprises adhesive properties, wherein the adhesive properties provide a means to attach the personal care article directly to the wearer's body.

6. The personal care article according to claim 1, wherein the shell has a three-dimensional concave shape such that the first side of the shell has an inward curving surface.

7. The personal care article according to claim 1, wherein the shell is a unitary structure.

8. The personal care article according to claim 1, further comprising an anterior portion and a posterior portion, wherein the posterior portion is adapted to attach to the body of a wearer between the vulva region and the coccyx of the body of the wearer and the anterior portion is adapted to attach to the mons veneris region of the wearer.

9. The personal care article according to claim 8, wherein the posterior portion further comprises a protrusion extending upward from the first side of the shell.

10. The personal care article according to claim 1, wherein the shell is breathable.

11. The personal care article according to claim 1, wherein the shell comprises material that is selected from extensible or elastically extensible materials.

12. The personal care article according to claim 1, wherein the absorbent structure is attached to the shell via an attachment means selected from at least one of a snap, a hook-and-loop material, a line of adhesive or a spot bond.

13. The personal care article according to claim 1, wherein the absorbent structure is at least partially free-floating.

14. The personal care article according to claim 1, wherein the absorbent structure is sized and shaped to cover the labia majora of a wearer.

15. The personal care article according to claim 1, wherein the absorbent structure further comprises at least one of a body-side liner, a backsheet, a surge layer or a transfer layer.

16. The personal care article according to claim 1, further comprising a release sheet.

17. The personal care article according to claim 1, wherein the shell includes at least one of a printed color, a print design, a texture, a micro-embossing, a printed message or a set of instructions.

18. The personal care article according to claim 1, further comprising a positioning aid.

19. The personal care article according to claim 1, further comprising a removal aid.

20. The personal care article according to claim 1, wherein the personal care article is selected from a pantiliner, a sanitary napkin, a maxi-pad or an incontinence article.

21. The personal care article according to claim 1, wherein the absorbent structure is integrated into the shell.

22. The personal care article according to claim 1, wherein the first area of the shell is liquid permeable and the second area of the shell is liquid impermeable.

23. The personal care article according to claim 1, wherein the shell comprises an absorbent material.

24. The personal care article according to claim 1, wherein the personal care article is part of a personal care system.

25. A personal care article comprising:
a shell for attachment to the vulva region of a female wearer;
wherein the shell has a first side and a second side, the first side having a first area and a second area;
wherein at least a portion of the first area comprises a body adhesive for adhesively attaching the shell directly to the wearer's skin and/or hair surrounding the vulva region;
wherein the second area is not directly attached to the wearer's skin, and wherein the second area provides a barrier between the vulva and the wearer's clothing when in use.

26. The personal care article according to claim 25, further comprising an attachment means for attaching an absorbent structure.

27. The personal care article according to claim 25, further comprising an absorbent material.

28. The personal care article according to claim 25, wherein the shell is selected from a nonwoven web, a gel, a film, a sheet of a polymeric material or a foam.

29. The personal care article according to claim 25, wherein the shell is breathable and liquid-impermeable.
